(19) United States
(12) Patent Application Publication
(10) Pub. No.: US 2018/0206461 A1
(43) Pub. Date: Jul. 26, 2018

(54) INHIBITING CTRP5 ACTION TO IMPROVE INSULIN RESISTANCE ASSOCIATED WITH OBESITY AND TYPE 2 DIABETES

(71) Applicant: THE JOHNS HOPKINS UNIVERSITY, BALTIMORE, MD (US)

(72) Inventors: GUANG WILLIAM WONG, LUTHERVILLE, MD (US); XIA LEI, JESSUP, MD (US)

(21) Appl. No.: 15/745,625

(22) PCT Filed: Jul. 15, 2016
(86) PCT No.: PCT/US16/42446

§ 371 Date: Jan. 17, 2018

Related U.S. Application Data
(60) Provisional application No. 62/193,792, filed on Jul. 17, 2015.

(51) Int. Cl. A01K 67/027; A61K 45/06; A61K 45/00

(52) U.S. Cl. CPC A01K 67/0276; A61K 45/06; A61K 45/00; A01K 2267/0362; A01K 2217/075; A01K 2227/105; A01K 2207/25

(57) ABSTRACT

The presently disclosed subject matter relates to methods of improving insulin sensitivity in cells, tissues, and subjects, as well as methods of reducing insulin resistance, improving glucose homeostasis, reducing hepatic tryglyceride levels, reducing food intake, and treating metabolic disorders, such as those occurring in individuals who are obese, diabetic, and/or have hepatic steatosis.

### CROSS-REFERENCE TO RELATED APPLICATIONS

This application claims the benefit of U.S. Provisional Application No. 62/193,792, filed Jun. 17, 2015, which is incorporated herein by reference in its entirety.

### FEDERALLY SPONSORED RESEARCH OR DEVELOPMENT

This invention was made with government support under DK084171 awarded by the National Institutes of Health. The government may have certain rights in the invention.

### INCORPORATION-BY-REFERENCE OF MATERIAL SUBMITTED ELECTRONICALLY

This application contains a sequence listing. It has been submitted electronically via EFS-Web as an ASCII text file entitled “111232-00540_ST25.txt”. The sequence listing is 24,576 bytes in size, and was created on Jul. 11, 2016. It is hereby incorporated by reference in its entirety.

### BACKGROUND

The gene that encodes CTRP5, a secreted protein of the C1q family, is mutated in individuals with late-onset retinal degeneration. CTRP5 is widely expressed outside the eye and also circulates in plasma. Its physiological role in peripheral tissues, however, has yet to be elucidated. Here, we show that Ctrp5 expression is modulated by fasting and refeeding, and by different diets, in mice. Adipose expression of CTRP5 was markedly upregulated in obese and diabetic humans, and in genetic and dietary models of obesity in rodents. Further, human CTRP5 expression in the subcutaneous fat depot positively correlated with BMI. A genetic loss-of-function mouse model was used to address the metabolic function of CTRP5 in vivo. On a standard chow diet, CTRP5-deficient mice had reduced fasting insulin but were otherwise comparable to wild-type littermate controls in body weight and adiposity. However, when fed a high-fat diet, CTRP5-deficient animals had reduced food intake, attenuated hepatic steatosis, and improved insulin action. Loss of CTRP5 also improved the capacity of chow-fed aged mice to respond to subsequent high-fat feeding, as evidenced by decreased insulin resistance. In cultured adipocytes and myotubes, recombinant CTRP5 treatment attenuated insulin-stimulated AKT phosphorylation. Our results provide the first genetic and physiological evidence for CTRP5 as a negative regulator of glucose metabolism and insulin sensitivity. Inhibition of CTRP5 action may result in the alleviation of insulin resistance associated with obesity and diabetes.

C1q/TNF-related proteins (CTRP1-15) are a highly conserved family of secreted plasma proteins with a shared signature C1q globular domain (Seldin, et al., 20141; Wong, et al., 2004). They are widely expressed in human and mouse tissues and have important metabolic functions (Wong, et al., 2004; Peterson, et al., 2012; Peterson, et al., 2013; Peterson, et al., Am J Physiol Regul Integr Comp Physiol, 2013; Peterson, et al., 2010; Seldin, et al., 2013; Seldin, et al., 2012: Wei, et al., 2014; Wei, et al., 2012; Wei, et al., 2011; Wei, et al., 2013; Wong, et al., 2009; Wong, et al., 2008). CTRP5 is expressed by a variety of tissues, including the adipose tissue (Wong, et al., 2008; Schmid, et al., 2013) and retinal pigment epithelium and ciliary body of the eye (Mandal, et al., 2006); interestingly, an autosomal dominant missense mutation (S163R) in the CTRP5/C1QTNF5 gene causes late onset retinal degeneration (L-ORD) in humans (Hayward, et al., 2003; Ayyagari, et al., 2005; Subrayan, et al., 2005; Soumplis, et al., 2013; Vincent, et al., 2012). Functional and structural studies have revealed that Ser-163 plays a critical role in the protein's structural integrity, as mutation of this residue promotes protein aggregation and impairs the secretion and assembly of CTRP5 into proper higher-order oligomeric structures important for its biological function (Mandal, et al., 2006; Hayward, et al., 2003; Ayyagari, et al., 2005; Shu, et al., 2006; Tu, et al., 2014; Shu, et al., Adv Exp

Med Biol, 2006). Two targeted Ctrp5 S163R knock-in mutant mouse models have been generated to model the human disease. While one S163R knock-in mouse model recapitulates the phenotypes of human L-ORD (Chavali, et al., 2011), another knock-in mouse model, with a different genetic background, lacks discernable retinal defects (Shu, et al., 2011).

CTRP5 is detected beyond the visual system, in peripheral tissues such as adipose tissue. Its function in the periphery, however, remains uncertain. Several recent studies suggest a metabolic role for CTRP5. Notably, serum CTRP5 levels are higher in genetic models of obesity and diabetes (ob/ob and db/db mice, OLETF rat) (Park, et al., 2009). In obese Pima Indians, the expression of CTRP5 transcript is upregulated in isolated subcutaneous adipocytes relative to lean controls (Lee, et al., 2005). In healthy female volunteers, serum CTRP5 levels decreased after a 10-week aerobic exercise regimen and were positively correlated with insulin resistance index (HOMA-IR) (Lim, et al., 2012). However, in a different study involving a larger cohort of non-diabetic male and female volunteers, combined aerobic and resistance exercise for 12 weeks modestly increased serum CTRP5 levels (Choi, et al., 2013). In cultured myocytes, CTRP5 expression and secretion is increased when mitochondrial DNA is depleted, and recombinant CTRP5 treatment appears to enhance fatty acid oxidation (Park, et al., 2009). In cultured adipocytes, recombinant CTRP5 treatment also inhibits the secretion of adipokines such as resistin and adiponectin (Schmid, et al., 2013).

Despite these in vitro observations and correlative studies in humans, the physiological role of CTRP5 in peripheral tissues remains elusive.

### SUMMARY

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting escriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml.

In an effort to illuminate the role of CTRP5 in modulating metabolic function, the presently disclosed subject matter employed a genetic loss-of-function mouse model to help elucidate the role of CTRP5 in vivo.

In an aspect, the presently disclosed subject matter provides a method of improving insulin sensitivity in a cell, tissue, or subject, the method comprising administering to a cell, tissue, or subject an effective amount of an agent that decreases the expression level and/or activity of C1q and tumor necrosis factor related protein 5 (CTRP5), thereby improving insulin sensitivity in the cell, tissue, or subject.

In an aspect, the presently disclosed subject matter provides a method of reducing insulin resistance in a subject in need thereof, the method comprising administering to the subject an effective amount of an agent that decreases the expression level and/or activity of CTRP5, thereby reducing food intake in the subject.

In an aspect, the presently disclosed subject matter provides a method of improving glucose homeostasis in a subject in need thereof, the method comprising administering to the subject an effective amount of an agent that decreases the expression level and/or activity of CTRP5, thereby reducing food intake in the subject.

In an aspect, the presently disclosed subject matter provides a method of reducing food intake in a subject in need thereof, the method comprising administering to the subject an effective amount of an agent that decreases the expression level and/or activity of CTRP5, thereby reducing food intake in the subject.

In an aspect, the presently disclosed subject matter provides a method of reducing hepatic tryglycerides in a subject in need thereof, the method comprising administering to the subject an effective amount of an agent that decreases the expression level and/or activity of CTRP5, thereby reducing hepatic tryglycerides in the subject.

In an aspect, the presently disclosed subject matter provides a method of treating hepatic steatosis in a subject in need thereof, the method comprising administering to the subject an effective amount of an agent that decreases the expression level and/or activity of CTRP5, thereby treating hepatic steatosis in the subject.

In an aspect, the presently disclosed subject matter provides a method of treating a metabolic disorder in a subject in need thereof, the method comprising administering to the subject an effective amount of an agent that decreases the expression level and/or activity of CTRP5, thereby reducing food intake in the subject.

In an aspect, the presently disclosed subject matter provides a method of treating a metabolic disorder in a subject in need thereof, the method comprising administering to the subject an effective amount of an agent that decreases the expression level and/or activity of CTRP5, wherein the agent reduces insulin resistance, improves glucose homeostasis, reduces hepatic triglyceride levels, and/or reduces food intake in the subject, thereby treating a metabolic disorder in the subject.

In particular embodiments, the expression level and/or activity of CTRP5 is decreased in the cell, tissue, or subject. In particular embodiments, the expression level and/or activity of CTRP5 is decreased in a cell or tissue of the subject.

In particular embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In particular embodiments, the cell is selected from the group consisting of an adipocyte, a myocyte, a hepatocyte and combinations thereof. In particular embodiments, the tissue comprises a peripheral tissue. In particular embodiments, the tissue is selected from the group consisting of adipose tissue, skeletal muscle, liver, and combinations thereof. In particular embodiments, the adipose tissue comprises subcutaneous white adipose tissue.

In particular embodiments, the subject has a metabolic disorder. In particular embodiments, the subject is obese or at risk of becoming obese. In particular embodiments, the subject is obese or at risk of becoming obese and exhbits insulin resistance. In particular embodiments, the subect is obese or at risk of becoming obese and has diabetes (e.g., Type II diabetes). In particular embodiments, the subject has diabetes or is at risk of developing diabetes. In particular embodiments, the subject: (i) has a metabolic disorder or is at risk of developing a metabolic disorder; (ii) is obese or at risk of becoming obese; (iii) has hepatic steatosis; and/or (iv) has diabetes or is at risk of developing diabetes. In particular embodiments, the subject is refed in a fasted state. In particular embodiments, the subject is refed following a fast. In particular embodiments, the method includes administering to the subject a high fat diet. In particular embodiments, the subject is administered a high fat diet in a fasted state. In particular embodiments, the method includes administering to the subject an effective amount of an anti-diabetic agent. In particular embodiments, the method includes administering to the subject an effective amount of an appetite suppressant agent. In particular embodiments, the method includes administering to the subject an effective amount of an anti-diabetic agent and/or an appetite suppressant agent.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1A and FIG. 1B show evolutionary conservation of C1q/TNF-related protein 5 (CTRP5) in vertebrates and its tissue expression profile in humans. FIG. 1A shows sequence alignment of human (NP_001265360), mouse (NP_001177248), chicken (XP_001232467), frog (Xenopus; XP_002935065), and zebrafish (NP_001025124) CTRP5 using a web-based Clustal W (version 2) tool (Larkin, et al., 2007). Identical amino acids are shaded black and similar amino acids are shaded gray. Shading was done using the web-based BoxShade tool. The NH₂-terminal signal peptide, collagen domain (with G-X—Y repeats), and the COOH-terminal globular C1q domain are indicated. Sequences shown in FIG. 1A are in order from top to bottom: SEQ. ID. NO. 57 (human), SEQ. ID. NO. 58 (mouse), SEQ. ID. NO. 59 (chicken), SEQ. ID. NO. 60 (xenopus), SEQ. ID. NO. 61 (zebrafish). FIG. 1B shows quantitative real-time PCR analysis of human CTRP5 mRNA expression across 47 tissue types. Expression levels of CTRP5 in each tissue were normalized to GAPDH.

FIG. 2A, FIG. 2B, FIG. 2C, FIGS. 2D, and 2E show Ctrp5 expression in different metabolic states. FIG. 2A shows quantitative real-time PCR analysis of Ctrp5 expression in epididymal white adipose tissue (eWAT), skeletal muscle, liver, and hypothalamus of mice subjected to overnight fast (fasted group, N=7) or overnight fast followed by 3 h refeeding (refed group, N=8). Expression levels were normalized to β-actin. FIG. 2B and FIG. 2C show quantitative real-time PCR analysis of Ctrp5 expression in epididymal white adipose tissue (eWAT) from leptin-deficient ob/ob (N=10) and wild-type (WT) lean controls (N=9) or in eWAT and inguinal white adipose tissue (iWAT) from mice fed a control low-fat diet (LFD; N=8) vs. a high-fat diet (HFD; N=8). FIG. 2D shows expression levels of Ctrp5 in the brain and peripheral tissues of a separate cohort of LFD-fed (N=11) and HFD-fed (N=11) mice. Expression levels were normalized to β-actin. FIG. 2E shows quantitative real-time PCR analysis of Ctrp5 expression in brain, heart, liver, kidney, and eWAT from mice fed a ketogenic diet (N=8) or matched control diet (N=8). Expression levels were normalized to the average of 18s rRNA, Gapdh, β-actin and Rpl-22. *p<0.05, **<0.01, ***p<0.001.

FIG. 3A, FIG. 3B, FIG. 3C, and FIG. 3D show expression of CTRP5 in lean and obese humans. Quantitative real-time PCR analysis of CTRP5 in omental as shown in FIG. 3A and FIG. 3C, or subcutaneous as shown in FIG. 3B and FIG. 3D, adipose tissue of human abdominal surgery subjects. Expression of CTRP5 in the subcutaneous fat depot is positively correlated with body mass index (BMI) as shown in FIG. 3B. Expression levels of CTRP5 are higher in obese individuals with or without type 2 diabetes relative to lean individuals as shown in FIG. 3D (N=7-8). Expression levels were normalized to β-actin levels in each sample. **p<0.01

FIG. 4A, FIG. 4B and FIG. 4C show generation of Ctrp5-null mice. FIG. 4A is a schematic showing the strategy for generating Ctrp5 knockout (KO) mice. The entire Ctrp5 gene, comprising two exons, was replaced by a neomycin-resistance gene and lacZ reporter cassette. FIG. 4B is PCR genotyping results showing the successful generation of wild-type (WT; +/+), heterozygous (+/−), and homozygous KO (−/−) alleles using the indicated primer pairs (TUF and TUR for WT allele, laclnF, and laclnR for KO allele) shown in FIG. 4A. FIG. 4C shows the absence of Ctrp5 mRNA in eWAT from the knockout (KO) mice was confirmed by RT-PCR with primers specific for Ctrp5 (mCtrp5F and mCtrp5R).

FIG. 5A, FIG. 5B, FIG. 5C, FIG. 5D, FIG. 5E, FIG. 5F, and FIG. 5G show metabolic phenotypes of Ctrp5-null mice fed a standard laboratory chow diet. FIG. 5A shows body weight of wild-type (WT) and knockout (KO) male mice over time. FIG. 5B shows fat and lean mass in WT and KO mice quantified by Echo-MRI. FIG. 5C shows WT and KO blood glucose levels were measured at the indicated time points during glucose tolerance test (GTT). FIG. 5D shows WT and KO blood glucose levels were measured at the indicated time points during insulin tolerance test (ITT). FIG. 5E and FIG. 5F show fasting blood glucose and insulin levels. FIG. 5G shows calculated insulin resistance (HOMA-IR) index for WT and KO mice at 20 weeks of age. WT, N=7; KO, N=8. *p<0.05, **p<0.01.

FIG. 6A, FIG. 6B, FIG. 6C, FIG. 6D, FIG. 6E, FIG. 6F, FIG. 6G, FIG. 6H, FIG. 6I, FIG. 6J, FIG. 6K, and FIG. 6L show improved insulin sensitivity in Ctrp5-null mice fed a high-fat diet. FIG. 6A shows body weight of wild-type (WT) and knockout (KO) male mice over time. FIG. 6B shows fat and lean mass in WT and KO mice quantified by Echo-MRI at 21 weeks of age. FIG. 6C, FIG. 6D, FIG. 6E, and FIG. 6F show fasting blood glucose, insulin, and C-peptide levels as well as the calculated insulin resistance (HOMA-IR) index for WT and KO mice at 20 weeks of age. FIG. 6G shows real-time PCR for gluconeogenic gene (G6Pc and Pckl) expression in liver of WT and KO mice. FIG. 6H shows blood glucose levels for WT and KO mice were measured at the indicated time points during glucose tolerance test (GTT). FIG. 6I shows WT and KO serum insulin levels at 0 and 30 min after glucose injection. FIG. 6J shows WT and KO blood glucose levels measured at the indicated time point during insulin tolerance test (ITT). FIG. 6K shows the decay constant (K_{ITT}) for WT and KO mice based on the ITT data. FIG. 6L shows area-under-curve for ITT (as shown in FIG. 6J) was calculated for WT and KO mice. WT, N=8; KO, N=7. *p<0.05, **p<0.01.

FIG. 7A, FIG. 7B, and FIG. 7C show insulin-stimulated Akt phosphorylation in Ctrp5-null adipose tissue, skeletal muscle, and liver. Quantitative Western blot analysis of insulin-stimulated Akt (Ser⁴⁷³) phosphorylation in adipose tissue (FIG. 7A), skeletal muscle (FIG. 7B), and liver (FIG. 7C) of WT and KO mice injected with insulin (1 U/kg body WI). Tissues were harvested at 15 min post-insulin injection. A total of 10 μg protein lysate from each sample was loaded onto Western blot gels. *P<0.05.

FIG. 8A, FIG. 8B, FIG. 8C, FIG. 8D, FIG. 8E, FIG. 8F, FIG. 8G, FIG. 8H, FIG. 8I, and FIG. 8J show lipid and adipokine profiles of Ctrp5-null mice fed a high-fat diet. FIG. 8A shows representative histologic sections of liver from WT and KO mice stained with hematoxylin and eosin. FIG. 8B and FIG. 8C show liver triglyceride and cholesterol levels of WT and KO mice. FIG. 8D, FIG. 8E, FIG. 8F, and FIG. 8G show serum concentrations of triglycerides, cholesterol, and non-esterified free fatty acids (NEFA) and β-hydroxybutyrate (ketone) in WT and KO mice. FIG. 8G shows quantitative PCR analysis of genes involved in de novo lipid synthesis (Scdl , Fasn, Srebplc, and Accl) and fat oxidation (Lead and Mcad) in WT and KO mouse liver. FIG. 8H shows skeletal muscle triglyceride levels of WT and KO mice. FIG. 8I shows quantitative PCR analysis of genes involved in de novo lipid synthesis (Scdl, Fasn, Srebplc, and Accl) and fat oxidation (Lead and Mcad) in WT and KO mouse liver. FIG. 8J shows expression of genes (Gpat, Agpat, Dgat) involved in triglyceride synthesis in WT and KO mouse liver. All expression levels were normalized to 18s rRNA. WT, N=8; KO, N=7.

FIG. 9A, FIG. 9B, FIG. 9C, FIG. 9D, FIG. 9E, FIG. 9F, FIG. 9G, FIG. 9H, FIG. 9I, FIG. 9J, and FIG. 9K, show inflammatory and fibrotic states of adipose tissue in Ctrp5-null mice. FIG. 9A shows representative histologic sections of eWAT from WT and KO mice stained with hematoxylin and eosin. FIG. 9B and FIG. 9C show quantitative PCR analysis of macrophage marker genes (F4/80 and Cd11) in visceral (epididymal; eWAT) and subcutaneous (inguinal; iWAT) white adipose tissue. FIG. 9D and FIG. 9E show expression levels of fibrotic collagen genes (Col3 and Col6) in the visceral (eWAT) and subcutaneous (iWAT) fat depots of WT and KO mice. FIG. 9F, FIG. 9G, FIG. 9H, and FIG. 9I, show ELISA quantification of serum leptin, adiponectin, IL-6 and TNF-α levels in WT and KO mice. FIG. 9J and FIG. 9K showexpression levels of adiponectin and CTRPs in the eWAT and iWAT of WT and KO mice. All expression levels were normalized to 18s rRNA levels. WT, N=8; KO, N=7.

FIG. 10A, FIG. 10B, FIG. 10C, FIG. 10D, FIG. 10E, FIG. 10F, FIG. 10G, and FIG. 10H show indirect calorimetry analysis of Ctrp5-null mice fed a high-fat diet. FIG. 10A, FIG. 10B, FIG. 10C, and FIG. 10D show oxygen consumption (V_{O2}), CO₂ production (V_{CO2}), respiratory exchange ratio (RER), and energy expenditure (EE) for male WT and KO mice at 22 weeks of age. FIG. 10E shows total physical activity levels for WT and KO mice during the dark and light phases of the photocycle. FIG. 10F shows real-time food intake measurements for WT and KO mice during the dark and light phases of the photocycle. FIG. 10G shows cumulative food intake (over a 12-h period) for WT and KO mice in the dark and light phases of the photocycle. *p<0.05 (WT, N=6; KO, N=8).

FIG. 11A, FIG. 11B, FIG. 11C, FIG. 11D, FIG. 11E, FIG. 11F, FIG. 11G, FIG. 11H, FIG. 11I, FIG. 11J, FIG. 11K, and FIG. 11L show reduced insulin resistance and hepatic triglyceride synthesis gene expression in aged Ctrp5-null mice fed a high-fat diet later in life. Weaned WT and KO male mice were fed a chow diet for 21 weeks and then a HFD for a 16 weeks. FIG. 11A shows body weights of male WT (N=9) and KO (N=7) mice after switching to a HFD. FIG. 11B, FIG. 11C, FIG. 11D, and FIG. 11E show fasting blood glucose, serum insulin, and C-peptide levels as well as the calculated insulin resistance (HOMA-IR) index of aged WT and KO mice after high-fat feeding for 16 weeks. FIG. 11F shows blood glucose levels of WT (N=7) and KO (N=5) mice at the indicated time points during glucose tolerance test (GTT). FIG. 11G shows serum insulin levels were measured in the same group of mice during GTT. FIG. 11H shows blood glucose levels of WT (N=7) and KO (N=5) mice at the indicated time points during insulin tolerance test (ITT). FIG. 11I shows the decay constant (K_{ITT}) for WT and KO mice based on the ITT data. FIG. 11J shows quantitative PCR analysis of genes involved in de novo lipid synthesis (Scdl, Fasn, Srebplc, and Accl) and fat oxidation (Lcad and Mcad) in WT and KO mouse liver. FIG. 11K shows expression of genes (Gpat, Agpat, and Dgat) involved in triglyceride synthesis in WT and KO mouse liver. Food was removed for 3 hours before liver tissue was harvested from mice. FIG. 11L shows area-under-curve for ITT (as shown in FIG. 10G) was calculated for WT and KO mice. Expression levels were normalized to 18s rRNA levels. WT, n=7; KO, n=6.*p<0.05, **p<0.01.

FIG. 12A, FIG. 12B, FIG. 12C, and FIG. 12D, shows recombinant mouse CTRP5 attenuates insulin-stimulated Akt phosphorylation. FIG. 12A and FIG. 12C show mouse 3T3-L1 adipocytes (FIG. 12A) and rat L6 myotubes (FIG. 12C) were treated overnight with control conditioned medium or conditioned medium containing recombinant mouse CTRP5. The following day, cells were washed once and then stimulated with vehicle control or 100 nM insulin for 5 min. Cell lysates were then subjected to Western blot analysis with total and phosphorylated Akt antibodies. FIG. 12B shows quantification of immunoblot results for 3T3-L1 adipocytes based on two independent experiments (N=4). FIG. 12C shows quantification of immunoblot results for L6 myotubes based on two independent experiments (N=4). *p<0.05; **p<0.01; ***p<0.001

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures.

Therefore, it is to be understood that the presently disclosed subject matter is not to be limited to the specific embodiments disclosed and that modifications and other embodiments are intended to be included within the scope of the appended claims.

The presently disclosed subject matter demonstrates that decreasing the expression level and/or activity of C1q and tumor necrosis factor related protein 5 (CTRP5) can improve insulin sensitivity in a cell, tissue, or subject. In addition, decreasing the expression level and/or activity of CTRP5 can also reduce insulin resistance, improve glucose homeostasis, and/or reduce food intake in a subject. The presently disclosed subject matter also provides a method of treating a metabolic disorder in a subject by administering an agent that decreases the expression level and/or activity of CTRP5. The presently disclosed subject matter further provides a method of treating hepatic steatosis in a subject in need thereof by administering an agent that decreases the expression level and/or activity of CTRP5.

### I. Methods of Improving Insulin Sensitivity in a Cell, Tissue, or Subject

In an aspect, the presently disclosed subject matter provides a method of improving insulin sensitivity in a cell, tissue, or subject, the method comprising administering to a cell, tissue, or subject an effective amount of an agent that decreases the expression level and/or activity of C1q and tumor necrosis factor related protein 5 (CTRP5), thereby improving insulin sensitivity in the cell, tissue, or subject. In some embodiments, the expression level and/or activity of CTRP5 is decreased in the cell, tissue, or subject.

As used herein, the term “insulin sensitivity” refers to the physiological condition in which cells respond to the normal actions of the hormone insulin. This is in contrast to insulin resistance, which is a physiological condition in which cells fail to respond to the normal actions of insulin. Accordingly, “improving insulin sensitivity” in a cell, tissue, or subject means an increase in insulin sensitivity in the cell, tissue, or subject of at least about 1%, 5%, 10%, 20%, 30%, 40%, 50%, 60%, 70%, 80%, 90%, or more, or as much as 100%, at least about a 2-fold, or at least about a 3-fold, or at least about a 4-fold, or at least about a 5-fold or at least about a 10- fold increase, or any increase between 2-fold and 10-fold or greater as compared to a reference level (e.g., the insulin sensitivity before employing the method and/or agent).

As used herein, “decreasing the expression level and/or activity of C1q and tumor necrosis factor related protein 5 (CTRP5)” includes any decrease in expression, protein activity, or level of the CTRP5 gene or protein encoded by the CTRP5 gene. The decrease may be at least 20%, 30%, 40%, 50%, 60%, 70%, 80%, 90%, 95% or 99% or more as compared to the expression of the CTRP5 gene or the activity or level of the CTRP5 protein. In some embodiments, the agent may inhibit the interaction of CTRP5 with a coactivator, cofactor, or another molecule that interacts with the CTRP5 gene and/or protein. In some embodiments, the agent may target a coactivator, cofactor, or another molecule that interacts with the CTRP5 gene and/or protein and/or is involved in the same pathway as the CTRP5 gene and/or protein to modulate insulin sensitivity.

In some embodiments, the agent that decreases the expression level and/or activity of CTRP5 is any therapeutic agent that is capable of decreasing the expression level and/or activity of CTRP5. In some embodiments, the agent is selected from the group consisting of small molecules, such as small organic or inorganic molecules; saccharides; oligosaccharides; polysaccharides; a biological macromolecule selected from the group consisting of peptides, proteins, peptide analogs and derivatives; peptidomimetics; nucleic acids, such as RNA interference molecules, selected from the group consisting of siRNAs, shRNAs, antisense RNAs, ribozymes, dendrimers and aptamers; antibodies, including antibody fragments and intrabodies; an extract made from biological materials selected from the group consisting of bacteria, plants, fungi, animal cells, and animal tissues; naturally occurring or synthetic compositions; and any combination thereof. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. Non-limiting examples of agents that decrease the expression level and/or activity of CTRP5 include CTRP5 siRNA (GeneCards Human Gene Database, http://www.genecards.org/cgi-bin/carddisp.pl?gene=C1QTNF5; CTRP5 siRNA (h): sc-77053, Santa Cruz Biotechnology, Inc., http://datasheets.scbt.com/sc-77053.pdf) and CTRP5 antibodies (GeneCards Human Gene Database, http://www.genecards.org/cgi-bin/carddisp.pl?gene=C1QTNF5; Anti-human CTRP5 (NT), Universal Biologicals, http://www.universalbiologicals.com/anti-human-ctrp5-nt-49180), the references of which are incorporated herein by reference.

As used herein, the term “small molecule” can refer to agents that are “natural product-like,” however, the term “small molecule” is not limited to “natural product-like” agents. Rather, a small molecule is typically characterized in that it contains several carbon—carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

As used herein, an “RNA interference molecule” refers to an agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. The succession of letters used as abbreviations for bases) that biochemically characterizes a specific nucleic acid. A polynucleotide sequence presented herein is presented in a 5′ to 3′ direction unless otherwise indicated.

The term “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

In some embodiments, the cell is selected from the group consisting of an adipocyte, a cell that primarily comprises adipose tissue; a myocyte, a type of cell found in muscle tissue; a hepatocyte, a cell comprising the main parenchymal tissue of the liver; and combinations thereof. In some embodiments, the cell is a skeletal muscle cell. In some embodiments, the cell is cardiomyocyte, a type of cell found in heart muscle tissue.

In some embodiments, the tissue comprises a peripheral tissue. As used herein, the term “peripheral tissue” refers to all the tissue in the body other than tissue in the central nervous system, such as liver, adipose, skeletal muscle, pancreatic tissue, kidney, etc. In some embodiments, the tissue is selected from the group consisting of adipose tissue, loose connective tissue comprising mostly adipocytes; skeletal muscle, a form of striated muscle tissue; liver, and combinations thereof. In some embodiments, the adipose tissue comprises subcutaneous white adipose tissue.

The presently disclosed subject matter provides the first genetic and physiological evidence for CTRP5 as a negative regulator of glucose metabolism and insulin sensitivity. Accordingly, in some embodiments, the subject has a metabolic disorder, such as a disorder associated with obesity and/or diabetes. In some embodiments, the subject is obese or at risk of becoming obese. As used herein, the term “obese” refers to a subject with a body mass index (BMI) of at least 30 kg/m². In some embodiments, the subject has diabetes or is at risk of developing diabetes. As used herein, the term “diabetes” refers the group of metabolic diseases in which a subject has a high blood sugar level over a prolonged period of time if not treated. The term “diabetes” includes all kinds of diabetes, such as type 1 diabetes, type 2 diabetes, and gestational diabetes. In some embodiments, the subject is refed in a fasted state. The term “fasted state” refers to a state in which a subject does not eat any food. In some embodiments, the subject does not eat for at least 1 hour, 2 hours, 4 hours, 6 hours, 8 hours, 10 hours, 12 hours, or more.

II. Methods of Reducing Insulin Resistance, Improving Glucose Homeostasis, and/or Reducing Food Intake in a Subject

In some embodiments, the presently disclosed subject matter provides a method of reducing insulin resistance in a subject in need thereof, the method comprising administering to the subject an effective amount of an agent that decreases the expression level and/or activity of CTRP5, thereby reducing insulin resistance in the subject. In some embodiments, “reducing insulin resistance” in a cell, tissue, or subject means a decrease in insulin resistance in the cell, tissue, or subject of at least about 1%, 5%, 10%, 20%, 30%, 40%, 50%, 60%, 70%, 80%, 90%, or more.

In some embodiments, the presently disclosed subject matter provides a method of improving glucose homeostasis in a subject in need thereof, the method comprising administering to the subject an effective amount of an agent that decreases the expression level and/or activity of CTRP5, thereby improving glucose homeostasis in the subject. As used herein, the term “glucose homeostasis” refers to the balance of insulin and glucagon to maintain blood glucose. “Improving glucose homeostasis” refers to the ability of a subject to better maintain blood glucose levels. In some embodiments, “improving glucose homeostasis” in a subject means an improvement of glucose homeostasis in the subject of at least about 1%, 5%, 10%, 20%, 30%, 40%, 50%, 60%, 70%, 80%, 90%, or more.

It has been found that decreasing the expression level and/or activity of CTRP5 in a subject can reduce the amount of food that a subject eats. Accordingly, in some embodiments, the presently disclosed subject matter provides a method of reducing food intake in a subject in need thereof, the method comprising administering to the subject an effective amount of an agent that decreases the expression level and/or activity of CTRP5, thereby reducing food intake in the subject. In some embodiments, “reducing food intake” in a subject means a decrease in food intake in the subject of at least about 1%, 5%, 10%, 20%, 30%, 40%, 50%, 60%, 70% or more.

III. Methods of Treating Hepatic Steatosis and/or Reducing Hepatic Triglycerides in a Subject

In some embodiments, the presently disclosed subject matter provides a method of reducing hepatic triglycerides in a subject in need thereof, the method comprising administering to the subject an effective amount of an agent that decreases the expression level and/or activity of CTRP5, thereby reducing triglycerides in the subject. In some embodiments, “reducing hepatic triglycerides” in a subject means a decrease in hepatic triglycerides in the subject of at least about 1%, 5%, 10%, 20%, 30%, 40%, 50%, 60%, 70%, 80%, 90%, or more. In some embodiments, reduction of hepatic tryclerides in the subject ameliorates hepatic insulin resistance in the subject.

In some embodiments, the presently disclosed subject matter provides a method of treating hepatic steatosis in a subject in need thereof, the method comprising administering to the subject an effective amount of an agent that decreases the expression level and/or activity of CTRP5, thereby treating hepatic steatosis in the subject. In some embodiments, the agent decreases the level of hepatic triglycerides in the subject, thereby treating hepatic steatosis in the subject.

### IV. Methods of Treating a Metabolic Disorder in a Subject

The presently disclosed subject matter also provides a method of treating a metabolic disorder in a subject in need thereof, the method comprising administering to the subject an effective amount of an agent that decreases the expression level and/or activity of CTRP5, thereby treating the metabolic disorder in the subject. In some embodiments, the agent reduces insulin resistance, improves glucose homeostasis, reduces hepatic tryglyceride levels, and/or reduces food intake in the subject.

In some embodiments, the expression level and/or activity of CTRP5 is decreased in a cell or tissue of the subject. In some embodiments, the cell is selected from the group consisting of an adipocyte, a myocyte, a hepatocyte and combinations thereof. In some embodiments, the tissue comprises a peripheral tissue. In some embodiments, the tissue is selected from the group consisting of adipose tissue, skeletal muscle, liver, and combinations thereof In some embodiments, the adipose tissue comprises subcutaneous white adipose tissue. In some embodiments, the agent is selected from the group consisting of small molecules, such as small organic or inorganic molecules; saccharides; oligosaccharides; polysaccharides; a biological macromolecule selected from the group consisting of peptides, proteins, peptide analogs and derivatives; peptidomimetics; nucleic acids, such as RNA interference molecules, selected from the group consisting of siRNAs, shRNAs, antisense RNAs, ribozymes, dendrimers and aptamers; antibodies, including antibody fragments and intrabodies; an extract made from biological materials selected from the group consisting of bacteria, plants, fungi, animal cells, and animal tissues; naturally occurring or synthetic compositions; and any combination thereof. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the subject has a metabolic disorder or is at risk of developing a metabolic disorder. In some embodiments, the subject is obese or at risk of becoming obese. In some embodiments, the subject has diabetes or is at risk of developing diabetes. In some embodiments, the subject is refed following a fast. In some embodiments, the method further comprises administering to the subject a high fat diet in a fasted state. In some embodiments, a “high fat” diet is a diet in which at least 60% kilocalories are derived from fat. In some embodiments, the subject has hepatis steatosis. In some embodiments, hepatic triglyceride synthesis is increased in the subject. In some embodiments, the subject has elevated liver triglyceride levels.

In some embodiments, the method further comprises administering to the subject an effective amount of an anti-diabetic agent, such as an agent that treats diabetes, lowers blood glucose, as well as an agent that is an appetite suppressant and/or anti-obesity drug, which can lower the risk of developing diabetes and/or treat diabetes. Examples of agents include, but are not limited to, insulin (rapid acting, intermediate acting, or long acting); insulin sensitizers, such as metformin; thiazolidinediones (TZDs), such as rosiglitazone and pioglitazone, which bind to PPARγ, a type of nuclear regulatory protein involved in transcription of genes regulating glucose and fat metabolism; secretagogues, such as tolbutamide, acetohexamide, tolazamide, chlorpropamide, glipizide, glyburide, glimepiride, gliclazide, glycopyramide, gliquidone, repaglinide, and nateglinide, which increase insulin output from the pancreas; alpha-glucosidase inhibitors, such as miglitol, acarbose, and voglibose, which slow the digestion of starch in the small intestine; injectable glucagon-like peptide analogs and agonists, such as exenatide, liraglutide, taspoglutide, and lixisenatide, which bind to a membrane glucagon-like peptide (GLP) receptor; dipeptidyl peptidase-4 inhibitors, such as vildagliptin, sitagliptin, saxagliptin, linagliptin, alogliptin, and septagliptin, which increase the blood concentration of the incretin GLP-1; and amylin agonist analogues, such as pramlintide, which slow gastric emptying and suppress glucagon.

In some embodiments, the method further comprises administering to the subject an effective amount of an appetite suppressant agent. Examples of appetite suppressant agents include, but are not limited to, phentermine, diethylpropion, oxymetazoline, benfluorex, butenolide, cathine, diethylpropion, FG-7142, phenmetrazine, phentermine, phenylpropanolamine, pyroglutamyl-histidyl-glycine, sibutramine, amphetamine, benzphetamine, bupropion, bupropion, dextroamphetamine, dexmethylphenidate, glucagon, methylenedioxypyrovalerone, liraglutide, lorcaserin, lisdexamfetamine dimesylate, methamphetamine, methylphenidate, phendimetrazine, phentermine, phenethylamine, and topiramate.

As used herein, the term “inhibit,” and grammatical derivations thereof, refers to the ability of an agent to block, partially block, interfere, decrease, or reduce the expression level and/or activity of CTRP5. Thus, one of ordinary skill in the art would appreciate that the term “inhibit” encompasses a complete and/or partial decrease in the expression level and/or activity of CTRP5, e.g., a decrease by at least 10%, in some embodiments, a decrease by at least 20%, 30%, 50%, 75%, 95%, 98%, and up to and including 100%.

As used herein, the term “treating” can include reversing, alleviating, inhibiting the progression of, preventing or reducing the likelihood of the disease, disorder, or condition to which such term applies, or one or more symptoms or manifestations of such disease, disorder or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the target tissue, and the like.

The term “combination” is used in its broadest sense and means that a subject is administered at least two agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the presently disclosed agents described herein can be administered alone or in combination with adjuvants that enhance stability of the agents, facilitate administration of pharmaceutical compositions containing them in certain embodiments, provide increased dissolution or dispersion, increase inhibitory activity, provide adjunct therapy, and the like, including other active ingredients.

The timing of administration of a presently disclosed agent and at least one additional therapeutic agent can be varied so long as the beneficial effects of the combination of these agents are achieved. Accordingly, the phrase “in combination with” refers to the administration of a presently disclosed agent and at least one additional therapeutic agent (e.g., anti-diabetic agent and/or appetite suppressant agent) either simultaneously, sequentially, or a combination thereof. Therefore, a subject administered a combination of a presently disclosed agent and at least one additional therapeutic agent can receive the presently disclosed agent and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the presently disclosed agent and at least one additional therapeutic agent are administered simultaneously, they can be administered to the subject as separate pharmaceutical compositions, each comprising either a presently disclosed agent or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a presently disclosed agent and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index(SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In therapeutic and/or diagnostic applications, the agents of the disclosure (e.g., agents that decrease the expression level and/or activity of CTRP5, anti-diabetic agents, anti-obesity agents, and/or appetite suppressant agents) can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the agents of the present disclosure, in particular, those formulated as solutions, may be administered parenterally, such as by intravenous injection. The agents can be formulated readily using pharmaceutically acceptable carriers well known in the art into dosages suitable for oral administration. Such carriers enable the agents of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these agents may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl- cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed agents.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Summary

Human tissue samples—Subcutaneous and visceral (omental) adipose tissues were obtained from the adipose biology core of the NIH-funded Mid-Atlantic NORC (Nutrition Obesity Research Center) at the University of Maryland. Study protocols were approved by the Institutional Review Board for Human Subjects Research at the University of Maryland. Informed consent was obtained from all human subjects. Type 2 diabetes mellitus is defined for subjects having a hemoglobin A1c value of 6.5 or greater according to the World Health Organization criteria (Alberti, et al., 1998). Characteristics of the lean (non-T2D), obese (non-T2D), and obese (with T2D) individuals are presented in Table 1. Fasting serum glucose, cholesterol, triglyceride, HDL, and LDL levels were not collected from the non-diabetic control individuals.

TABLE 1
Characteristics of lean (nondiabetic), obese
nondiabetic, and obese diabetic groups

| | | Obese Non-T2D | Obese T2D |
|---|---|---|---|
| | Lean (n = 8) | (n = 8) | (n = 7) |
| Age | 49.1 ± 15.2 | 32.8 ± 8.1 | 44.0 ± 8.2 |
| Sex (male or female) | 8 females | 8 females | 5 females, 2 |
| | | | males |
| BMI, kg/m² | 24.0 ± 1.1 | 47.7 ± 7.3 | 44.9 ± 3.8 |
| Height, cm | 167.6 ± 7.7 | 167.1 ± 7.0 | 168.8 ± 7.6 |
| Weight, kg | 78.1 ± 29.6 | 132.5 ± 17.0 | 127.5 ± 19.4 |
| Fasting glucose, mg/dl | | 90.9 ± 9.1 | 170.3 ± 66.4 |
| Cholesterol, mg/dl | | 160.5 ± 21.3 | 185.6 ± 53.8 |
| Triglyceride, mg/dl | | 77.9 ± 26.1 | 124.3 ± 35.3 |
| HDL, mg/dl | | 43.6 ± 9.3 | 40.6 ± 10.4 |
| LDL, mg/dl | | 101.23 ± 18.1 | 109.3 ± 33.0 |

Values are means ± SE.
T2D, type 2 diabetes.

Mice—Eight-week-old leptin-deficient ob/ob male mice and C57BL/6J male mice were obtained from The Jackson Laboratory (Bar Harbor, Me.). Mouse tissues were collected from fasted and re-fed experiments. For the fasted group, food was removed for 16 h (beginning at 10 h into the light cycle), and mice were euthanized at 2-3 h into the light cycle. For the refed group, mice were fasted for 16 h and re-fed with chow pellets for 3 h before being euthanized. Due to the randomness of food intake, an ad libitum-fed group was not included in the fasting and refeeding studies. To generate the diet-induced obesity model, four-week-old C57BL/6J male mice were fed a high-fat diet (HFD) (60% kcal derived from fat; D12492; Research Diets, New Brunswick, N.J.) or a control low-fat diet (LFD) (10% kcal derived from fat; D12450B; Research Diets, New Brunswick, N.J.) for 12 weeks. A separate cohort of male mice were also exposed to a ketogenic diet or a matched control diet for a period of 12 weeks (beginning at 8 weeks old) as previously described (Ellis, et al., 2015). All mice were housed in polycarbonate cages under a 12:12-h light:dark photocycle and had access to water ad libitum throughout the study period. All animal experiments were approved by the Animal Care and Use Committee of the Johns Hopkins University School of Medicine.

Ctrp5 knockout mice—The Ctrp5 (C1qtnf5) null mouse strain was created from ES cell clone 12534A-H11, obtained from the KOMP Repository (www.komp.org) and generated by Regeneron Pharmaceuticals (Tarrytown, N.Y.). Genotyping primers for the Ctrp5 wild-type (WT) allele were: TUF, 5′-CAGAAACCCTGATGCCTC TACTC-3′ (SEQ. ID. NO. 1) and TUR, 5′-GGAGAAATTAGGAGCC GCAGAAG-3′ (SEQ. ID. NO. 2). Primers for the knockout (KO) allele were: Laclnf, 5′-GGTAAACTGGCTCGGATTAGG G-3′ (SEQ. ID. NO. 3) and LaclnR, 5′-TTGACTGTAGCGGCTGATG TTG-3′(SEQ. ID. NO. 4). Ctrp5 KO mice were generated on a C57BL/6 genetic background. Unless otherwise noted, mice were fed ad libitum a standard laboratory chow diet (No. 5001, Lab Diet, St. Louis, Mo.). Body weights of Ctrp5 WT and KO mice were measured weekly. At the end of the studies, tissues were collected after the mice were euthanized. Epididymal white adipose tissue (eWAT), inguinal white adipose tissue (iWAT), liver, and skeletal muscle were quickly removed, snap-frozen in liquid nitrogen for RNA and protein extraction, or prepared for histological study. Blood samples were collected for serum analysis.

Glucose and insulin tolerance tests—Glucose tolerance tests (GTT) and insulin tolerance tests (ITT) were performed on Ctrp5 WT and KO mice fed chow diet or HFD for 16-20 weeks. For the GTT, mice were fasted for 6 h before intraperitoneal (i.p.) injection of lg glucose/kg body weight (BW). Blood was collected via tail bleed before and 30 min after injection, and glucose concentrations were measured using a glucometer (BD Biosciences, San Jose, Calif.) at 0, 15, 30, 60, and 120 min. For the ITT, food was removed 2 h before i.p. injection of 1U insulin/kg BW. Blood glucose concentrations were measured at 0, 15, 30, 45, 60, and 90 min. The homeostatic model assessment of insulin resistance (HOMA-IR) was calculated based on fasting glucose and insulin concentrations as HOMA-IR=(fasting glucose [mM] x fasting insulin [microunits/mL])/22.5 (Mattews, et al., 1985).

RNA isolation and real-time PCR analysis—Total RNA was isolated using Trizol reagent (Life Technologies, Carlsbad, Calif.) and 2 μg of RNA was reverse transcribed using GoScript™ Reverse Transcriptase (Promega, Madison, Wis.). 10 ng of cDNA from each sample were used in real-time PCR using SYBR® Green PCR master mix on a CFX Connect system (Bio-Rad Laboratories, Hercules, Calif.). Results were analyzed using the 2^{-ΔΔCt} method (Schmittgen, et al., 2008). Primer sequences are listed in Table 2. A ready-made human cDNA tissue panel (OriGene) was used to survey the tissue expression patterns of human CTRP5. To avoid detection of individual differences in gene expression, tissues were pooled from multiple individuals (based on the manufacturer's information); thus, each sample represented the average expression of CTRP5 in a particular tissue.

TABLE 2
Primers used in real-time PCR

| Gene | Forward (5=-3=) | Reverse (5=-3=) |
|---|---|---|
| Human | CCTCGCCTTTGCCGATCC (SEQ. ID. NO. 5) | CGCGGCGATATCATCATC (SEQ. ID. NO. 6) |
| f>-ACTIN | | |
| Human | CCCACCTGCAAAGTGAGCTCATG (SEQ. ID.NO. 7) | CTAGTCATTCACAATATTCCAG (SEQ. ID. NO. 8) |
| CTRP5 | | |
| 18s rRNA | GCAATTATTCCCCATGAACG (SEQ. ID. NO. 9) | GGCCTCACTAAACCATCCAA (SEO. ID. NO. 10) |
| Mouse | AGTGTGACGTTGACATCCGTA (SEQ. ID. NO. 11) | GCCAGAGCAGTAATCTCCTTCT (SEQ. ID. NO. 12) |
| f>-Actin | | |
| Mouse | AGCAGGTTTTGAAGTTCACCC (SEQ. ID. NO. 13) | CAGCTTTCCCATTCACCTTGA (SEQ. ID. NO. 14) |
| Rpl-22 | | |
| Mouse | TGGAGTCTGAGCCTCCGG (SEQ. ID. NO. 15) | AGAAGGGCAAGAAGTGGCC (SEQ. ID. NO. 16) |
| Ctrp5 | | |
| Scd1 | CCCAGTCGTACACGTCATTTT (SEQ. ID. NO. 17) | CATCATTCTCATGGTCCTGCT (SEQ. ID.NO. 18) |
| Fasn | GCTGCGGAAACTTCAGAAAAT (SEQ. ID. NO. 19) | AGAGACGTGTCACTCCTGGACTT (SEO. ID. NO. 20) |
| Srebp1c | GGAGCCATGGATTGCACATT (SEQ. ID. NO. 21) | GGCCCGGGAAGTCACTGT (SEQ. ID. NO. 22) |
| Acc1 | TGACAGACTGATCGCAGAGAAAG (SEQ. ID. NO. 23) | TGGAGAGCCCCACACACA (SEQ. ID. NO. 24) |
| Lcad | TCTTTTCCTCGGAGCATGACA (SEQ. ID. NO. 25) | GACCTCTCTACTCACTTCTCCAG (SEQ. ID. NO. 26) |
| Mcad | AGGGTTTAGTTTTGAGTTGACGG (SEQ. ID. NO. 27) | CCCCGCTTTTGTCATATTCCG (SEQ. ID. NO. 28) |
| Gpat1 | CAACACCATCCCCGACATC (SEO. ID. NO. 29) | GTGACCTTCGATTATGCGATCA (SEQ. ID. NO. 30) |
| Gpat3 | GGAGGATGAAGTGACCCAGA (SEQ. ID. NO. 31) | CCAGTTTTTGAGGCTGCTGT (SEQ. ID. NO. 32) |
| Gpat4 | TGTCTGGTTTGAGCGTTCTG (SEQ. ID. NO. 33) | TTCTGGGAAGATGAGGATGG (SEQ. ID. NO. 34) |
| Agpat1 | TAAGATGGCCTTCTACAACGGC (SEQ. ID. NO. 35) | CCATACAGGTATTTGACGTGGAG (SEQ. ID. NO. 36) |
| Agpat2 | CAGCCAGGTTCTACGCCAAG (SEQ. ID. NO. 37) | TGATGCTCATGTTATCCACGGT (SEQ. ID. NO. 38) |
| Agpat3 | CTGCTTGCCTACCTGAAGACC (SEQ. ID. NO. 39) | GATACGGCGGTATAGGTGCTT (SEQ. ID. NO. 40) |
| Agpat4 | CCAGTTTCTATGTCACCTGGTC (SEQ. ID. NO. 41) | GCAGAGTCTGGCATTGATCTTG (SEQ. ID. NO. 42) |
| Agpat6 | AGCTTGATTGTCAACCTCCTG (SEQ. ID. NO. 43) | CCGTTGGTGTAGGGCTTGT (SEQ. ID. NO. 44) |
| Dgat1 | CCCTGAGTATCCAGGCAAGG (SEQ. ID. NO. 45) | AAGGAGTGGGCCTCTAGACT (SEQ. ID. NO. 46) |
| Dgat2 | GCGCTACTTCCGAGACTACTT (SEQ. ID. NO. 47) | GGGCCTTATGCCAGGAAACT (SEQ. ID. NO. 48) |
| F4/80 | CCCCAGTGTCCTTACAGAGTG (SEQ. ID. NO. 49) | GTGCCCAGAGTGGATGTCT (SEQ. ID. NO. 50) |
| Cd11c | CTGGATAGCCTTTCTTCTGCTG (SEQ. ID. NO. 51) | GCACACTGTGTCCGAACTCA (SEQ. ID. NO. 52) |
| Col3 | GGGTTTCCCTGGTCCTAAAG (SEQ. ID. NO. 53) | CCTGGTTTCCCATTTTCTCC (SEO. ID. NO. 54) |
| Col6 | GATGAGGGTGAAGTGGGAGA (SEQ. ID. NO. 55) | CAGCACGAAGAGGATGTCAA (SEQ. ID. NO. 56) |

CTRP5, Clq/TNF-related protein 5;
Scd1, stearoyl-CoA desaturase;
Fasn, fatty acid synthase;
Srebp1c, sterol regulatoty element-binding protein-1c;
Acc1, acetyl-CoA carboxylase 1;
Lcad, long-chain acyl-CoA dehydrogenase;
Mcad, medium-chain acyl-CoA dehydrogenase;
Gpat; glycerol-3-phosphate acyltrans-ferase isoform;
Agpat, acylglycerolphosphate acyltransferase;
Dgat, diacylglycerol-acyltransferase.

Body composition analysis—Body composition of Ctrp5 WT and KO mice was determined using a quantitative nuclear magnetic resonance (NMR) instrument (Echo-MRI-100, Echo Medical Systems LLC, Waco, Tex.) at The Johns Hopkins University School of Medicine mouse phenotyping core facility. EcoMRI analyses measured fat mass, lean mass, and water content.

Indirect calorimetry—Ctrp5 WT and KO mice fed a HFD for 20 weeks were used for simultaneous assessments of daily body weight change, food intake (corrected for spillage), physical activity, and whole-body metabolic profile in an open-flow indirect calorimeter (CLAMS, Columbus Instruments, Columbus, Ohio). Data were collected for three days to confirm that mice were acclimated to the calorimetry chambers (indicated by stable body weights, food intakes, and diurnal metabolic patterns), and data were analyzed from the fourth day. Rates of oxygen consumption (VO₂) and carbon dioxide production (VCO₂) in each chamber were measured throughout the studies. Respiratory exchange ratio (RER=VCO₂/VO₂) was calculated by CLAMS software (version 4.02) to estimate relative oxidation of carbohydrates (RER=1.0) versus fats (RER=0.7), not accounting for protein oxidation. Energy expenditure (EE) was calculated as EE=VO₂×[3.815+(1.232×RER)]. VO₂, VCO₂, and EE data were normalized to lean body mass. Physical activities were measured by infrared beam breaks in the metabolic chamber. Average metabolic values were calculated per subject and averaged across subjects for statistical analysis.

Blood chemistry analysis—Tail vein or lateral saphenous vein blood samples were allowed to clot on ice and then centrifuged for 10 min at 10,000×g. Serum samples were stored at −80° C. Serum triglycerides and cholesterol were measured using an Infinity™ kit (Thermo Fisher Scientific, Middletown, Va.). Non-esterified free fatty acids were measured using a Wako kit (Wako Chemicals, Richmond, Va.). Serum insulin, leptin, adiponectin, IL-6, and TNF-α were measured by ELISA (Millipore, Billerica, Mass.) according to the manufacturer's instructions.

Recombinant CTRP5 production—Full-length mouse recombinant CTRP5, containing a C-terminal FLAG tag epitope, was produced in mammalian HEK293 cells (GripTite™, Invitrogen, Carlsbad, Calif.) as described previously (Wong, et al., 2008). Serum-free conditioned media containing recombinant CTRP5 was concentrated 25-fold, and the concentrated media was used in in vitro studies. Concentrated conditioned media from HEK293 cells transfected with control pcDNA3 plasmid was used as control.

Cell culture and Western blot analysis—Mouse 3T3-L1 adipocytes and rat L6 myocytes were cultured and differentiated as previously described (Wei, et al., 2013). For in vitro studies, differentiated 3T3-L1 adipocytes and L6 myotubes were cultured in 24-well plates. Cells were washed once with PBS, then incubated overnight in 180 μL of DMEM containing 0.5% BSA plus 20 μL of concentrated conditioned medium containing recombinant mouse CTRP5 or 20 μL of control conditioned media from cells transfected with pcDNA3 control plasmid. The following day, cells were washed once with PBS and incubated for 5 min in HEPES buffered saline solution (25 mM HEPES, pH 7.4, 120 mM NaCl, 5 mM KCl, 1.2 mM MgSO4, 1.3 mM CaCl2, 1.3 mM KH2PO4, and 0.5% BSA) containing 100 nM insulin or vehicle control. After that, medium was removed and cells were immediately lysed in lysis buffer (20 mM Tris-HCl, 150 mM NaCl, 1 mM EDTA, 0.5% NP-40, and 10% glycerol) with PhosSTOP phosphatase inhibitor cocktail (Roche, Basel, Switzerland) and protease inhibitor cocktail (Sigma-Aldrich, St. Louis, Mo.), boiled for 10 min in 95° C., and subjected to Western blot analysis using antibodies specific to AKT and phosphor-AKT (Ser-473) (Cell Signaling Technology, Beverly, Mass.). Western blots were carried out and quantified as previously described (Seldin, et al., 2012).

Histology—Formalin-fixed, paraffin-embedded white adipose tissue and liver sections were stained with hemotoxylin and eosin (HE) at the Pathology Core facility at The Johns Hopkins University School of Medicine. Images were captured with a Zeiss Axioplan upright microscope with a Zeiss Axiocam color CCD camera (Carl Zeiss Microscopy, Thornwood, N.Y.).

Statistical analysis—Kruskal—Wallis analysis of variance with pairwise comparisons was used to determine differences among the three human fat depot groups. Spearman's correlation coefficient analysis was used to analyze the associations between adipose expression of CTRP5 and BMI. Other comparisons were made using either a two-tailed Student's t-test for two groups or one-way ANOVA for multiple groups. Values reported are means±SEM. p<0.05 was considered statistically significant.

### Results

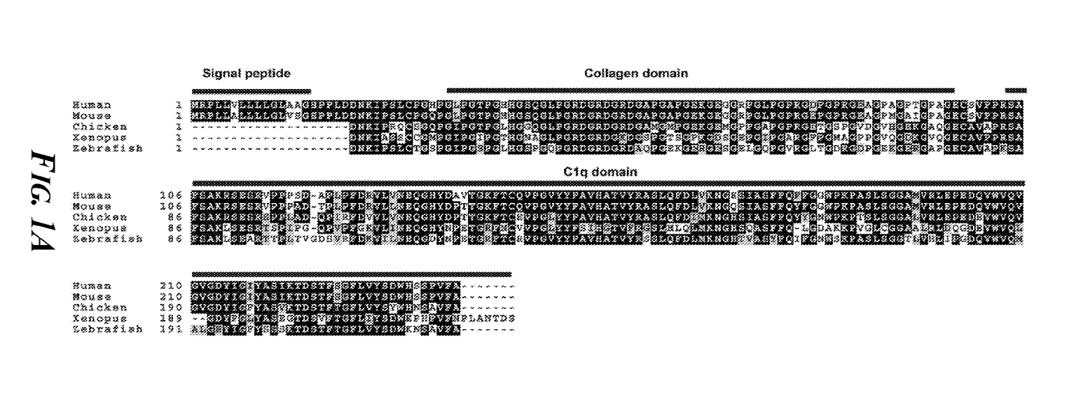

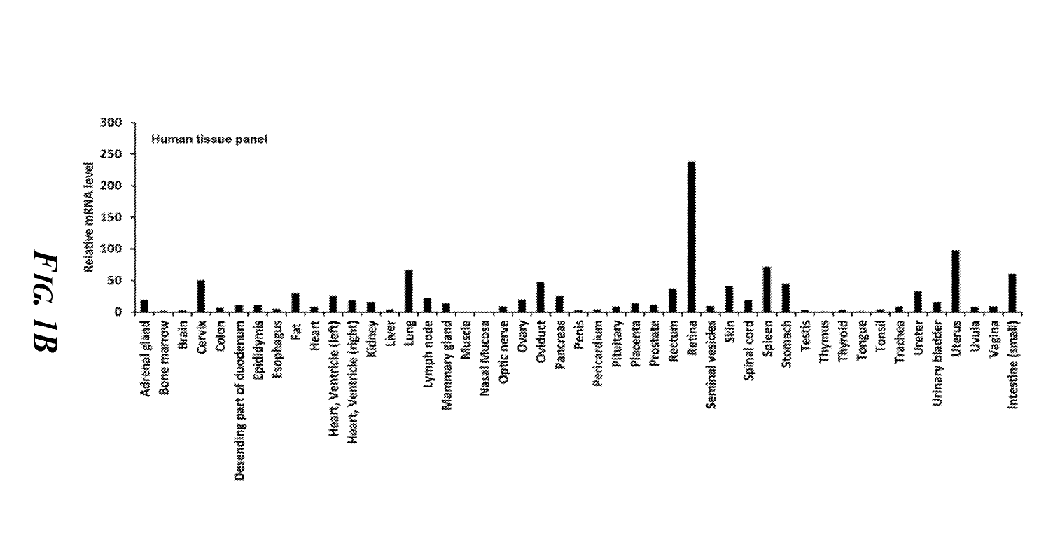

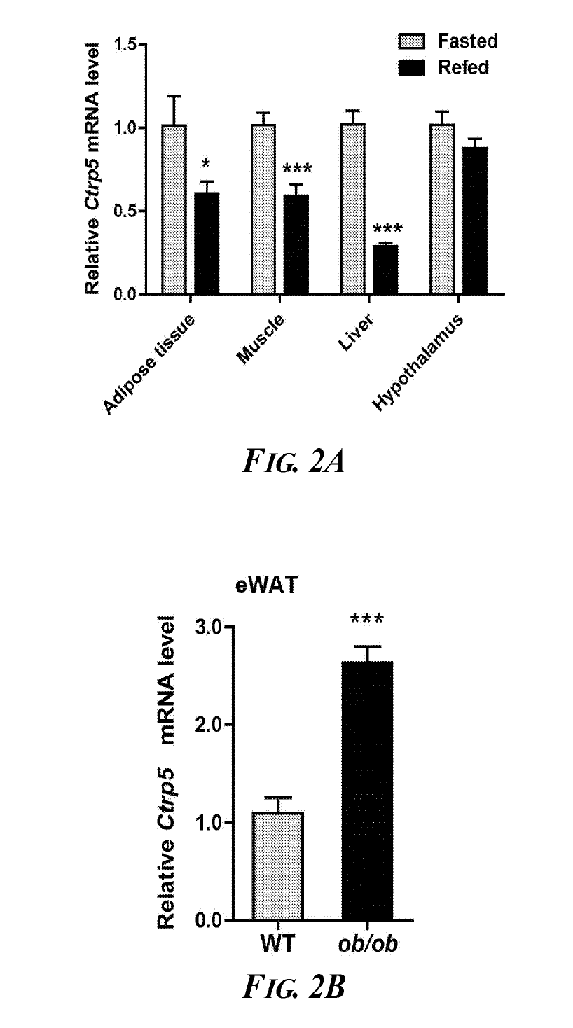

Expression of human CTRP5 in the peripheral tissues—Human and mouse CTRP5 are highly conserved, with similar protein domain structure (FIG. 1A), and the full-length proteins share 94% amino acid identity. A high degree of amino acid conservation also extends to chicken (Gallus gallus; 77%), clawed frog (Xenopus tropicalis; 61%), and zebrafish (Danio rerio; 65%) CTRP5. Expression profiling of human CTRP5 across 47 tissue types clearly indicated that the transcript is also widely expressed in peripheral tissue in addition to being most highly expressed in the retina (FIG. 1B), suggesting that it likely also has a function outside of the visual system.

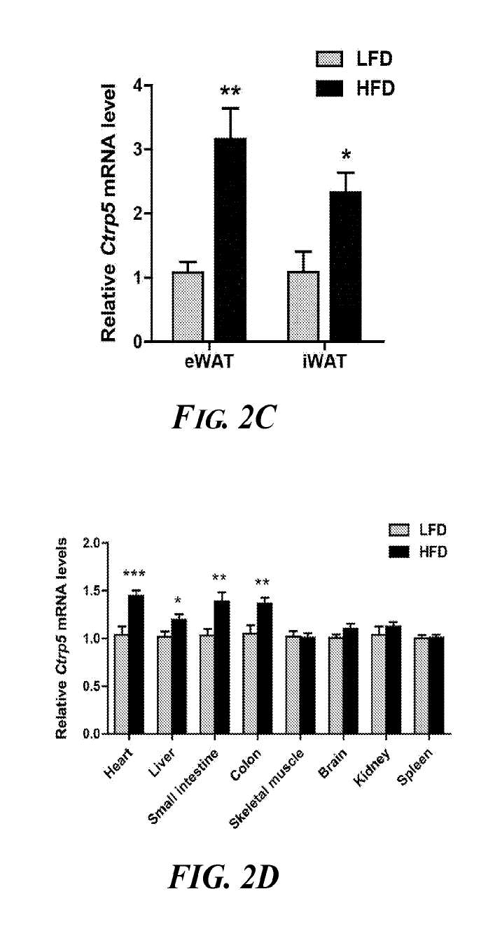

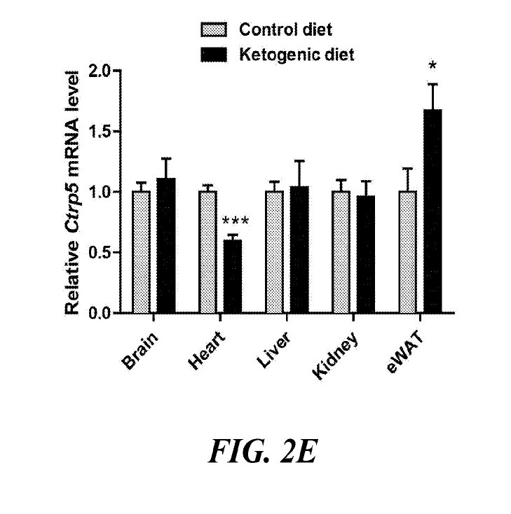

Expression of Ctrp5 in wild-type and obese mice—To explore the role of CTRP5 in metabolic response and energy homeostasis, we first measured the expression levels of Ctrp5 in several key metabolic tissues in wild-type C57BL/6J male mice under different metabolic states: either 16 h fast, or 16 h fast followed by 3 h refeeding. The expression levels of Ctrp5 were significantly reduced in epididymal white adipose tissue (eWAT), skeletal muscle, and liver, but were unchanged in the hypothalamus of refed mice compared to fasted animals (FIG. 2A). In genetic models of severe obesity, as in leptin-deficient ob/ob mice, Ctrp5 mRNA expression was markedly unregulated in eWAT (FIG. 2B), a change that parallels the increase in serum CTRP5 levels seen in these animals (Park, et al., 2009). In diet-induced obese (DIO) mouse models that more closely resemble human obesity, Ctrp5 expression was likewise significantly increased in both eWAT and iWAT of HFD-fed mice relative to animals that fed a control LFD (FIG. 2C).

Although both are high in fat content, HFD and ketogenic diet (low in carbohydrates) are known to elicit distinct effects on whole-body energy balance and lipid metabolism (Kennedy, et al., 2007; Jornayvaz, et al., 2010). For this reason, a separate cohort of mice was exposed to a ketogenic diet or an appropriately matched control diet. In the context of a ketogenic diet, but not HFD, the expression of Ctrp5 transcript was significantly and selectively reduced in the heart, but not brain, liver, or kidney (FIG. 2E). In contrast, adipose expression of Ctrp5 in ketogenic diet-fed mice was higher, but the magnitude of increase was much less compared to HFD-fed mice. These results indicate the acute and chronic metabolic state-dependent modulations of Ctrp5 expression in peripheral tissues.

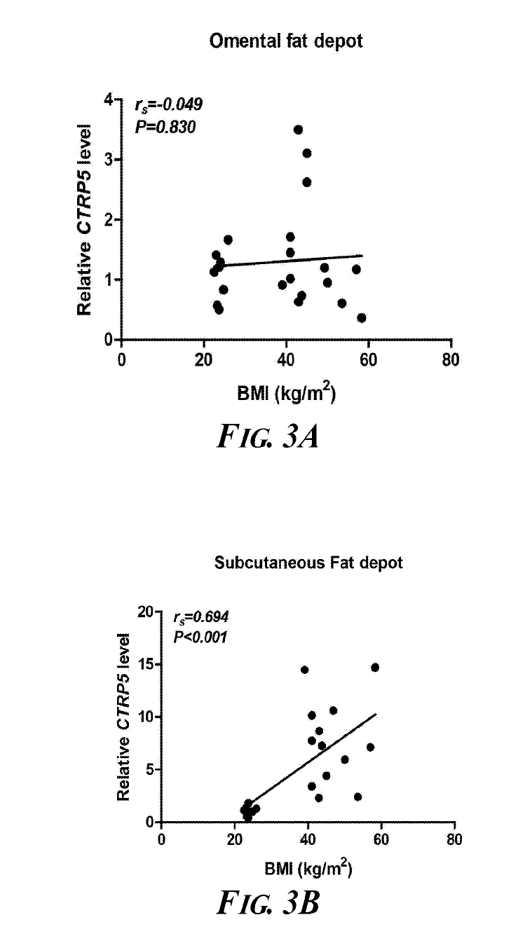

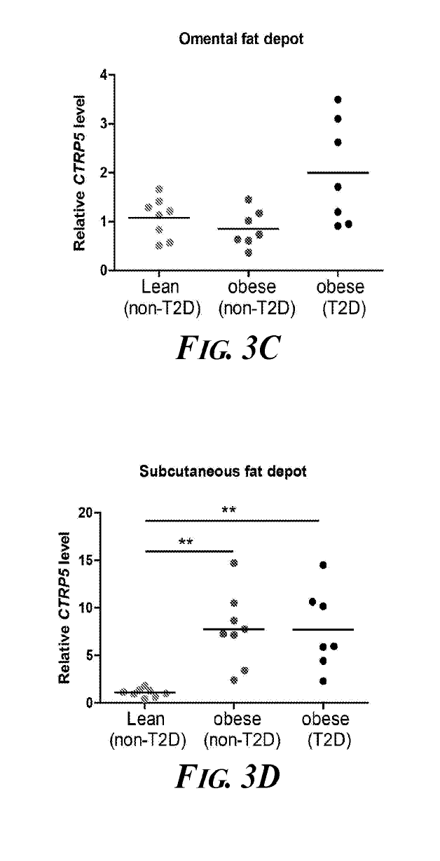

Expression of CTRP5 in humans—To address whether adipose expression of CTRP5 is also altered in human obesity, we measured its mRNA levels in subcutaneous and visceral (omental) fat depots. Expression of CTRP5 in subcutaneous, but not omental, fat depot was positively correlated with BMI (FIG. 3A and FIG. 3B). A marked increase in the expression of CTRP5 was observed in subcutaneous, but not omental, adipose tissue of obese non-diabetic and obese diabetic individuals when compared to lean controls (FIG. 3C and FIG. 3D).

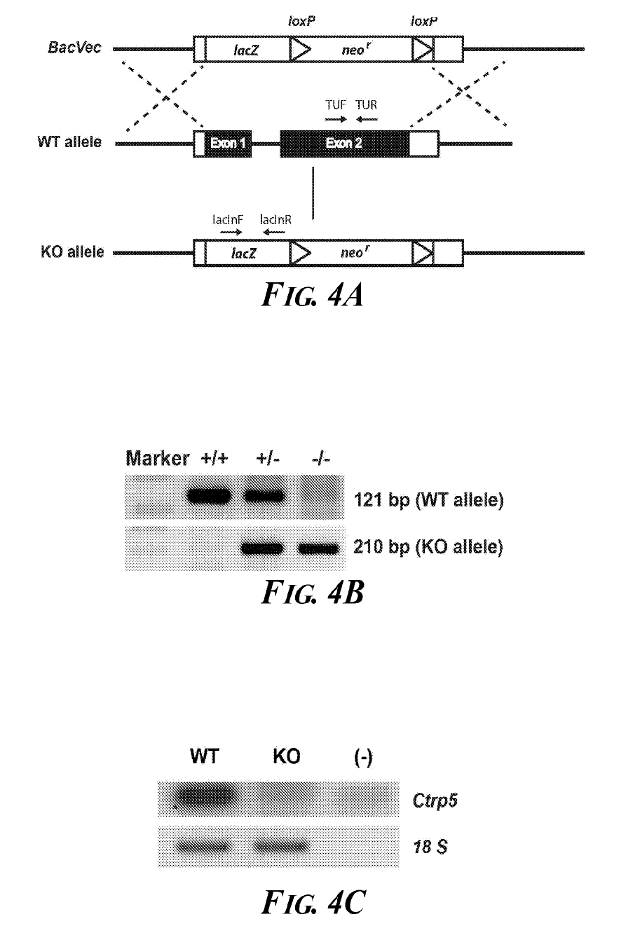

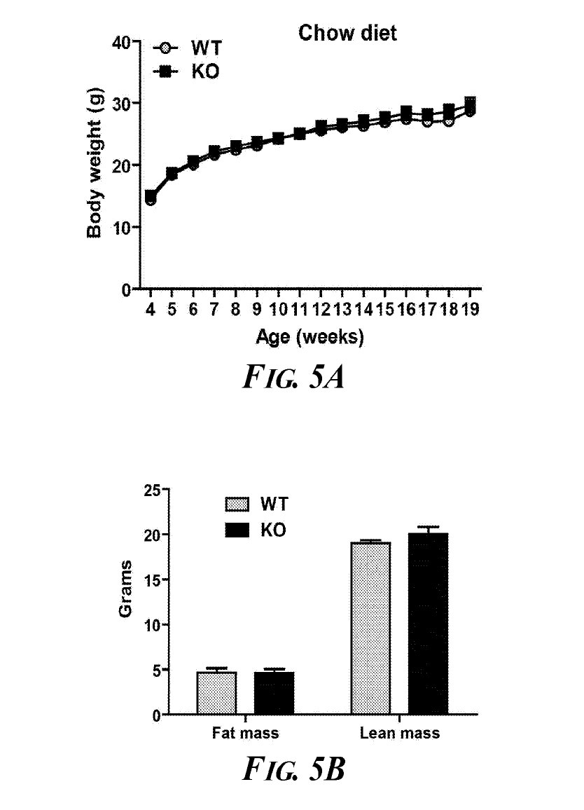

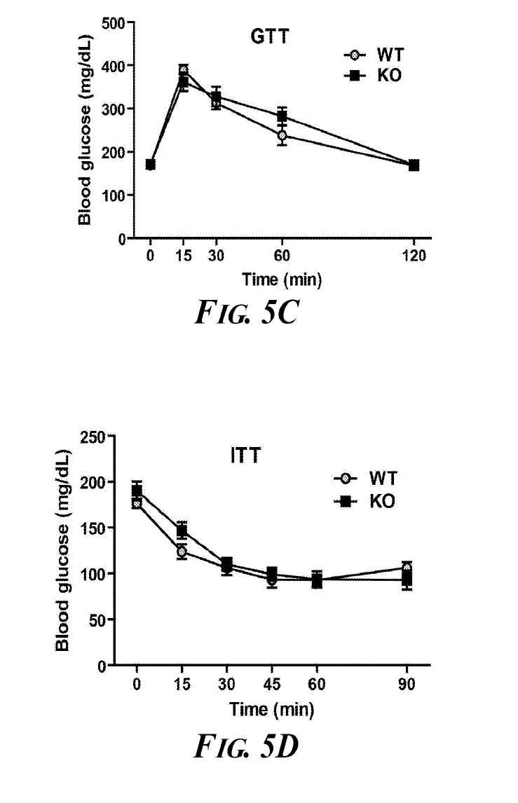

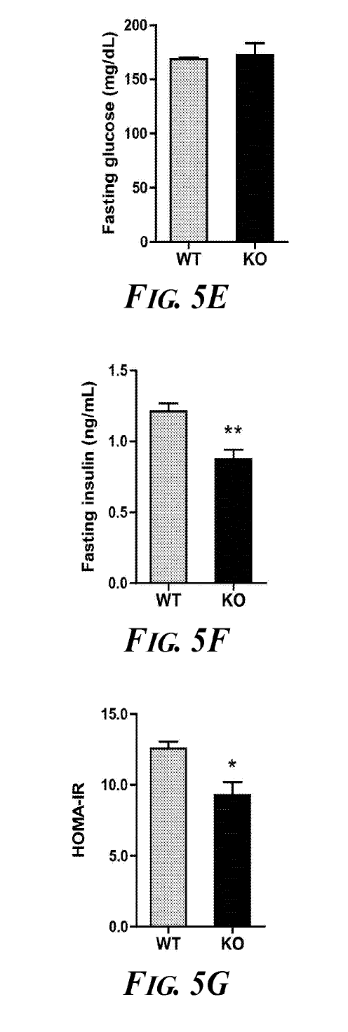

Generation of Ctrp5 knockout mice—A loss-of-function mouse model was used to establish the physiological role of Ctrp5. To generate Ctrp5-null mice, the entire gene, comprising two exons and one intron (1,090 bp on chromosome 9), was replaced with a targeting cassette containing a β-galactosidase reporter gene, lacZ (FIG. 4A). This strategy ensured that the KO mice were completely devoid of CTRP5 protein. Two sets of primers were designed to amplify a sequence within the protein coding region of the WT allele and a sequence spanning the upstream deletion site in the lacZ gene, respectively, to confirm the genotype of WT and KO mice (FIG. 4B). As expected, Ctrp5 mRNA was absent from the eWAT of KO mice (FIG. 4C). Ctrp5 KO mice were born with the expected Mendelian ratio and appeared normal with no gross developmental abnormalities (data not shown). Even though Ctrp5 is expressed throughout development, as early as embryonic day-7 (Wong, et al., 2008), it is dispensable for embryonic development in KO mice.

Metabolic phenotypes of Ctrp5 WT and KO mice fed a chow diet—To determine the contribution of Ctrp5 to systemic energy metabolism in the normal and pathophysiological context of diet-induced obesity, weaned 4-week-old Ctrp5 WT and KO mice were fed a standard laboratory chow or a HFD for 20 weeks. On a chow diet, we observed no differences in body weight, body composition (fat and lean mass), GTT, and ITT between female WT and KO mice (data not shown). Likewise, in male Ctrp5 WT and KO mice fed a chow diet, no differences were seen in body weight, fat and lean mass, glucose, and insulin tolerance (FIG. 5A, FIG. 5B, FIG. 5C, and FIG. 5D). While fasting blood glucose levels were not different between the two groups of mice (FIG. 5E), chow-fed Ctrp5 KO mice had significantly lower fasting insulin levels and insulin resistance index (HOMA-IR) (FIG. 5F and FIG. 5G).

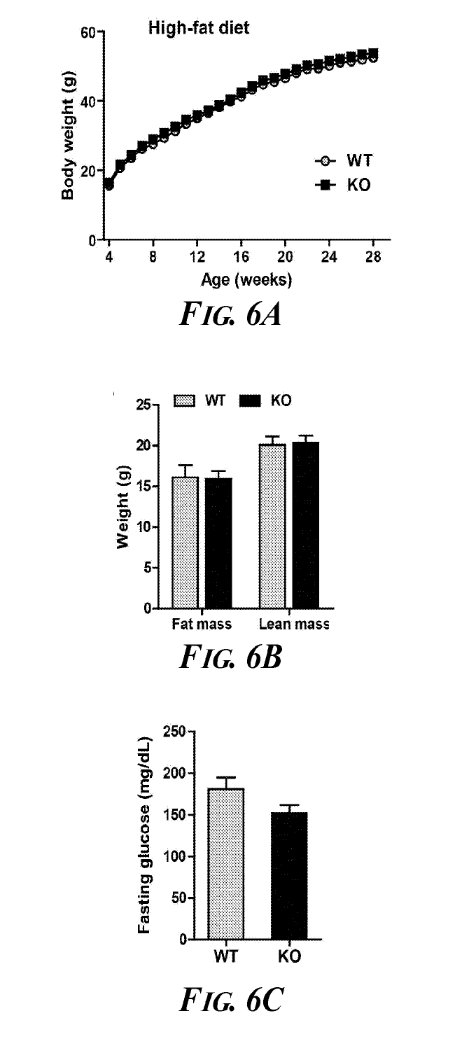

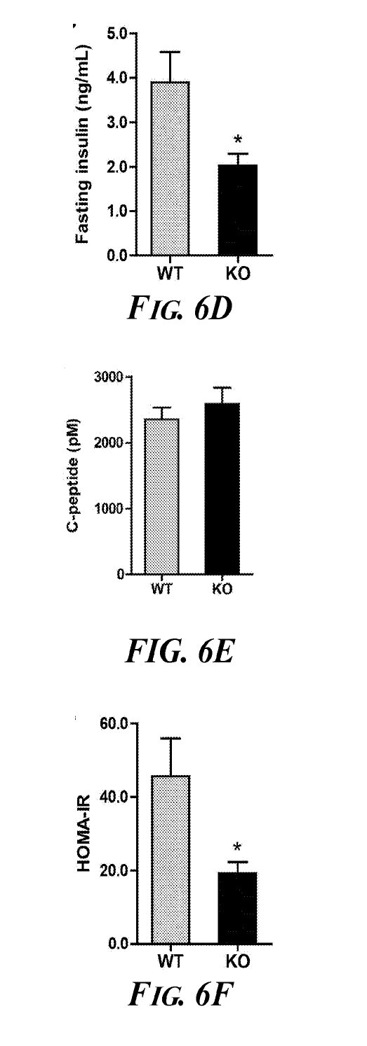

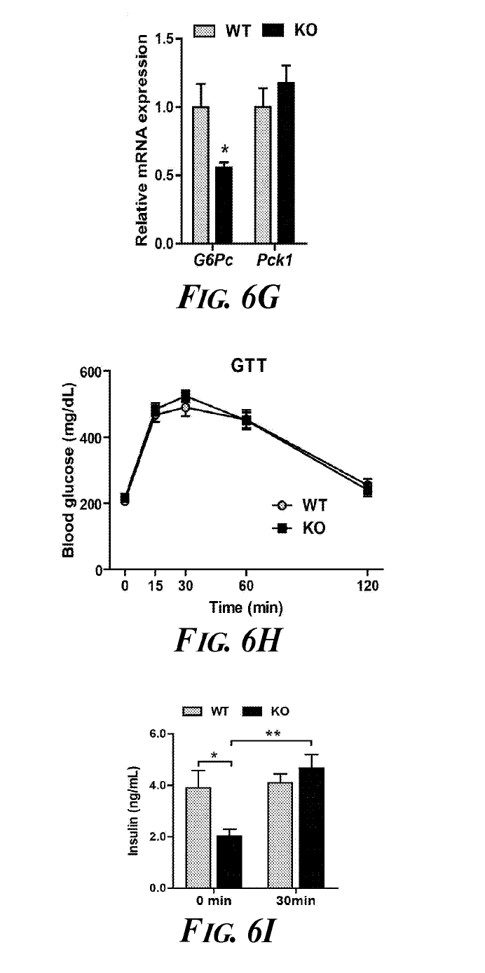

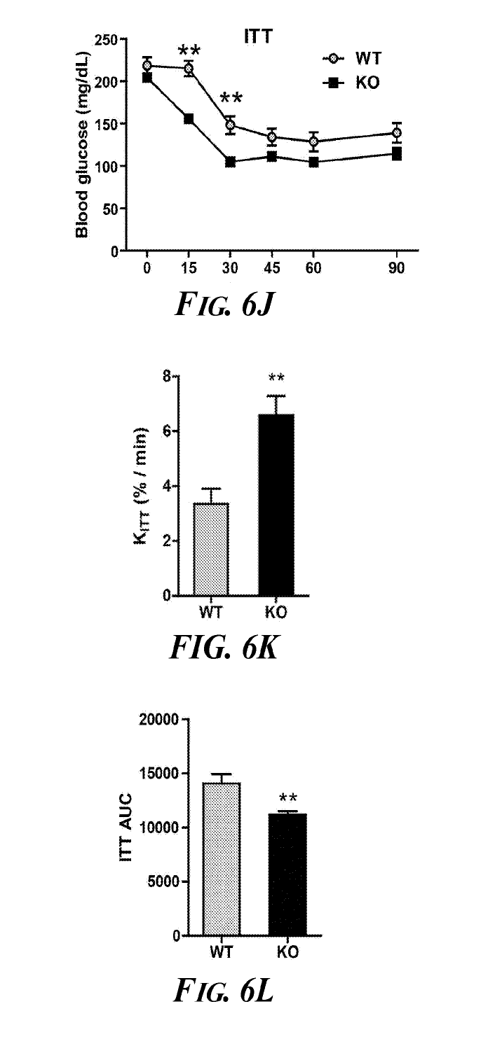

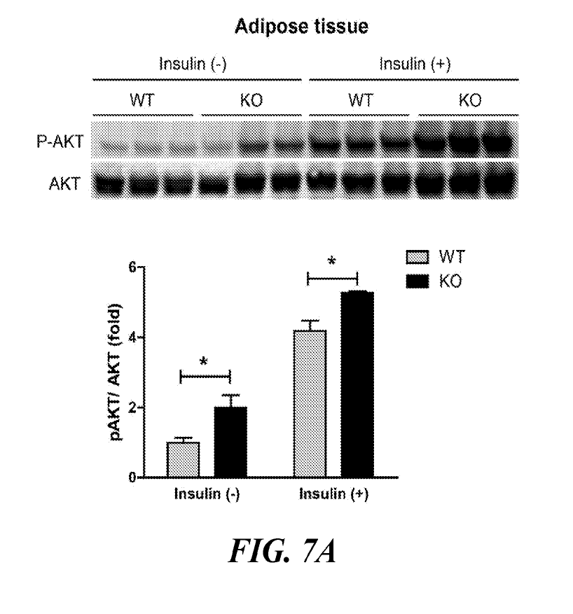

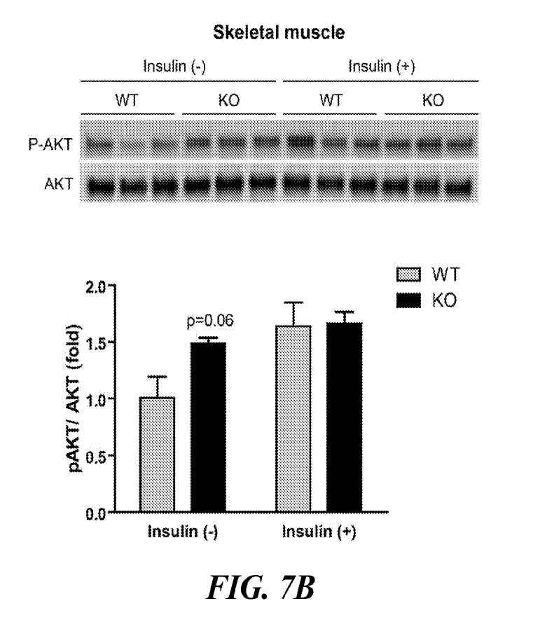

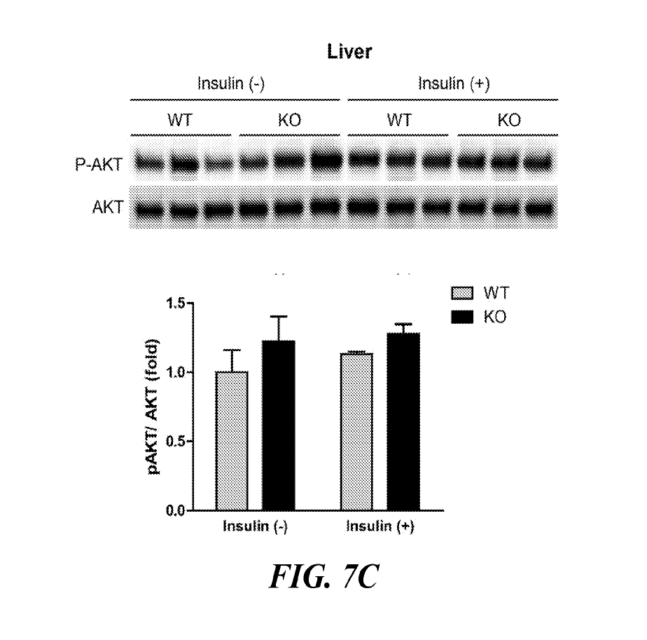

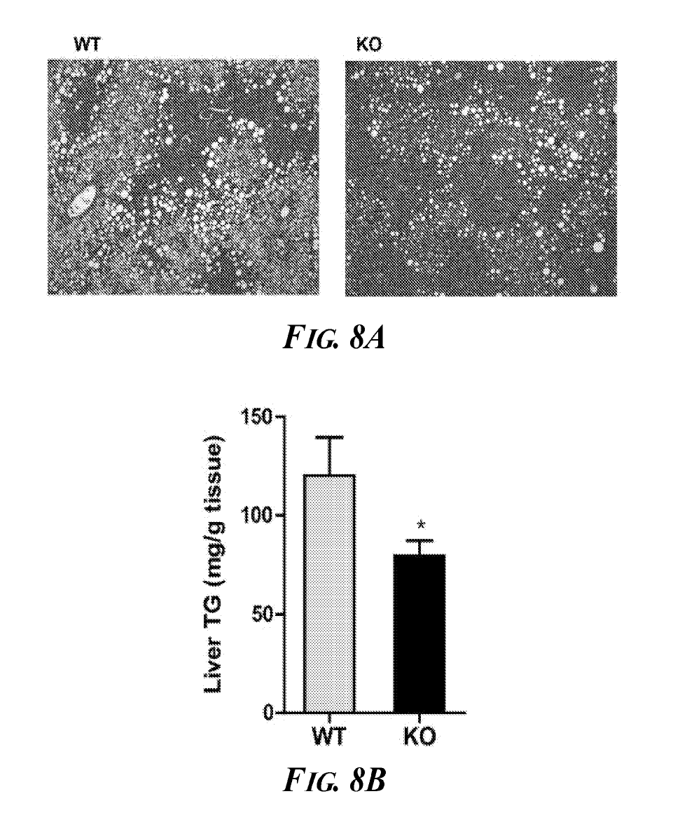

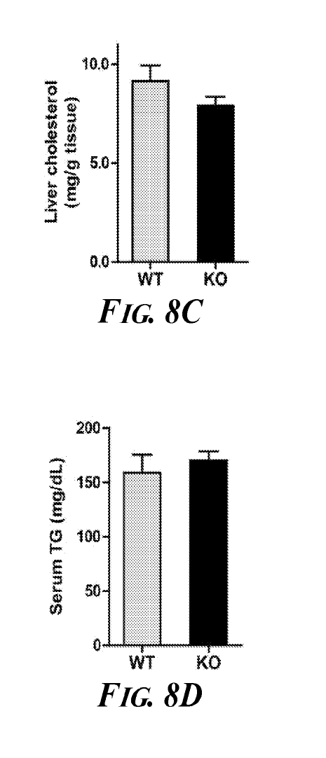

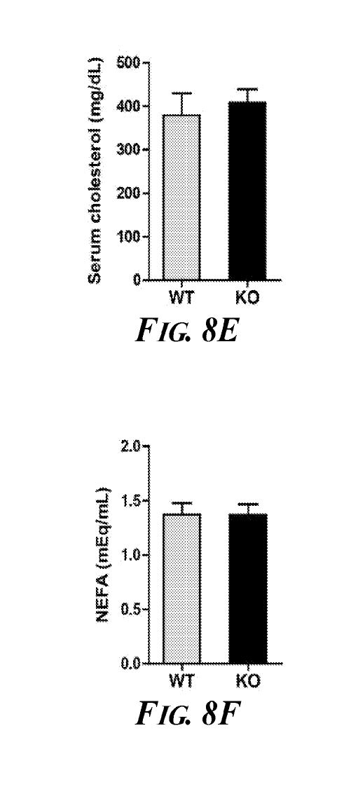

Improved insulin sensitivity in Ctrp5 KO mice fed a high-fat diet—We next examined the metabolic consequences of a calorically dense HFD in our mouse model. Similar to the chow-fed groups, we observed no differences in body weight, fat, or lean mass between HFD-fed Ctrp5 WT and KO mice (FIG. 6A and FIG. 6B). While fasting blood glucose levels were indistinguishable between the two groups, HFD-fed Ctrp5 KO mice had markedly lower fasting insulin levels and insulin resistance index (HOMA-IR), as well as reduced gluconeogenic gene (glucose-6-phosphatase, G6Pc) expression in the liver (FIG. 6C, FIG. 6D, FIG. 6F, and FIG. 6G). Despite seeing no differences in a glucose tolerance test (FIG. 6H), we observed robust insulin secretion in Ctrp5 KO mice in response to glucose challenge, while the insulin secretion profile was severely blunted in the obese HFD-fed WT mice (FIG. 61). When challenged with a bolus of insulin, the rate of insulin-stimulated glucose clearance in peripheral tissues was significantly greater in the Ctrp5 KO mice compared to WT controls (FIG. 6J, and FIG. 6L). Together, these results indicate that Ctrp5 deficiency improves whole-body insulin sensitivity in HFD-fed mice.

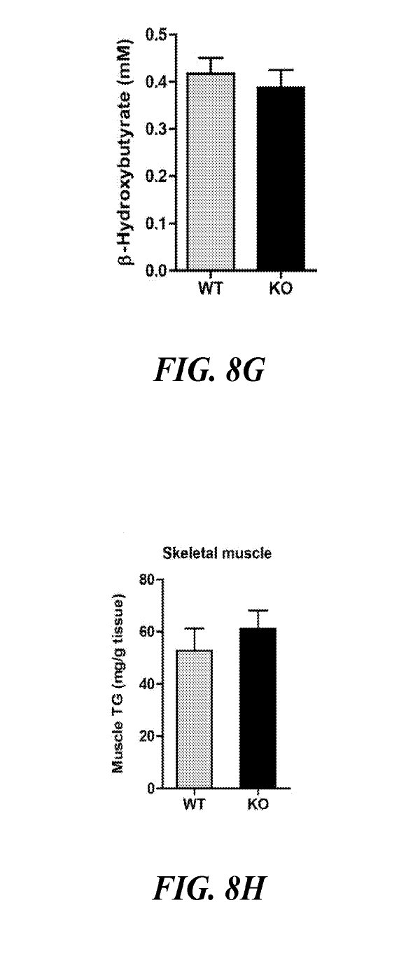

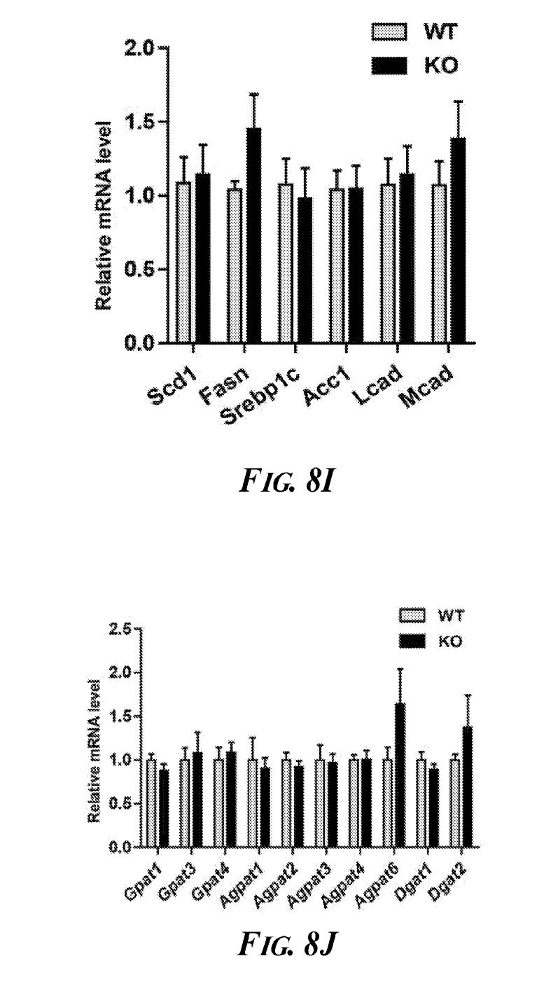

Lipid profiles in HFD-fed Ctrp5 KO mice—Excessive lipid accumulation in the liver, commonly known as hepatic steatosis, is frequently accompanied by insulin resistance. We performed histological analysis to determine whether changes in liver could account for the improvements in whole-body insulin resistance seen in the HFD-fed Ctrp5 KO mice. Hematoxylin and eosin staining showed that liver sections from KO mice had reduced steatosis compared to WT controls (FIG. 8A). In support of the histological data, we also observed reduced hepatic TG content, but not cholesterol levels, in the KO mouse liver relative to WT controls (FIG. 8B and FIG. 8C). Serum levels of TG, NEFA, and cholesterol, however, were not significantly different between the two groups (FIG. 8D, FIG. 8E, and FIG. 8F).

We next examined whether changes in the expression of hepatic lipid synthesis or fat oxidation genes might underlie the observed reduction in hepatic TG content. However, no differences were found in the expression of genes involved in de novo lipid synthesis (Scdl, Fasn, Srebplc, Accl), fat oxidation (Lcad, Mcad), or TG synthesis (Gpat, Agpat, and Dgat family members) between WT and KO mice (FIG. 8I and FIG. 8J).

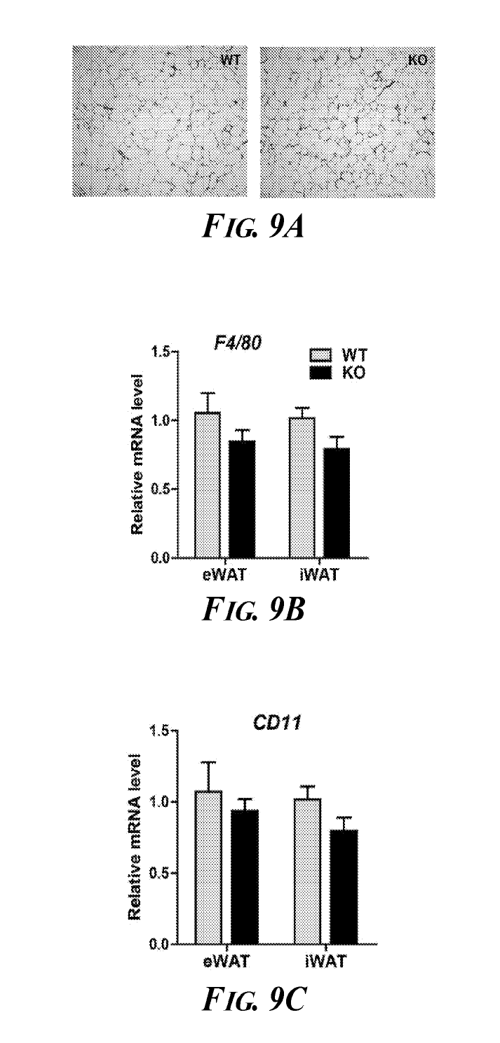

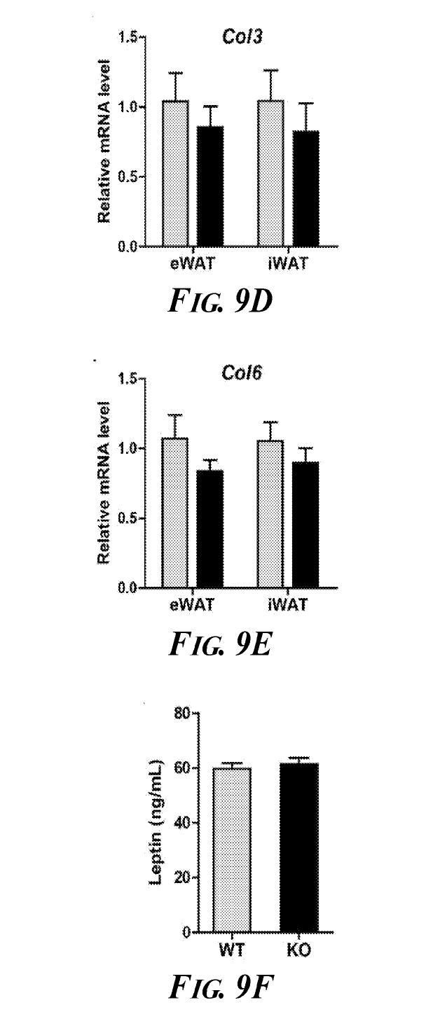

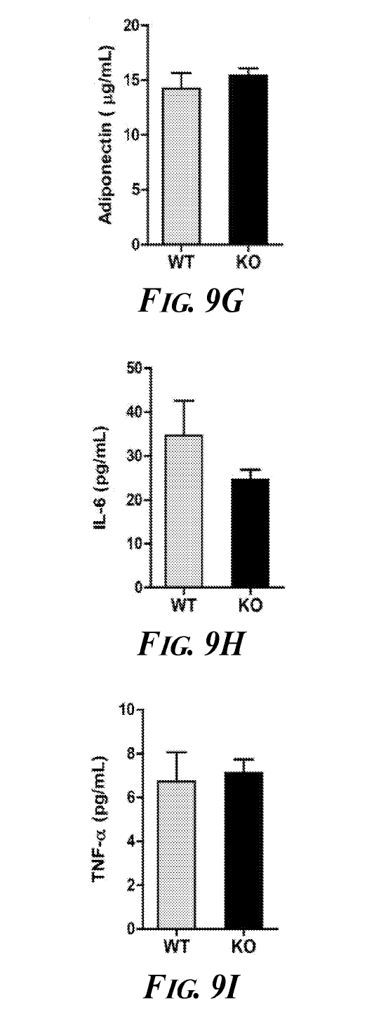

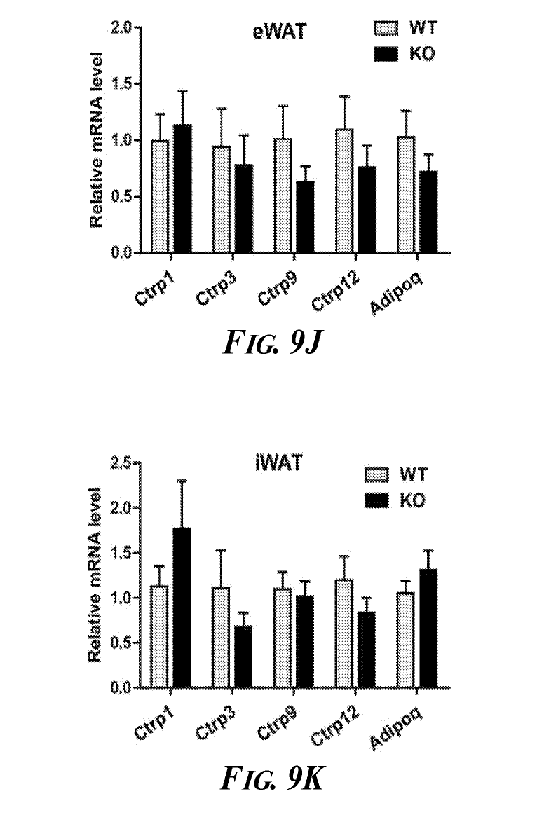

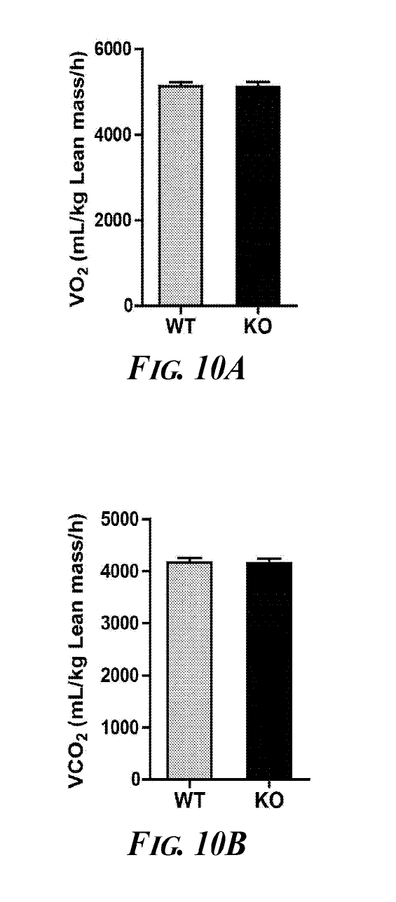

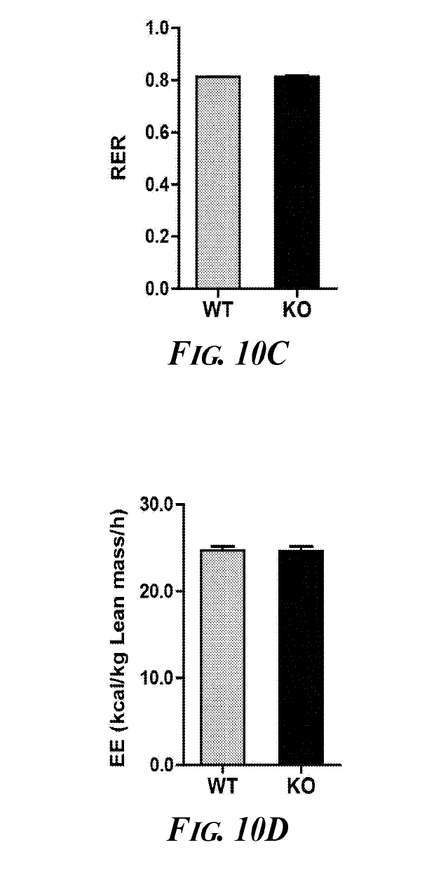

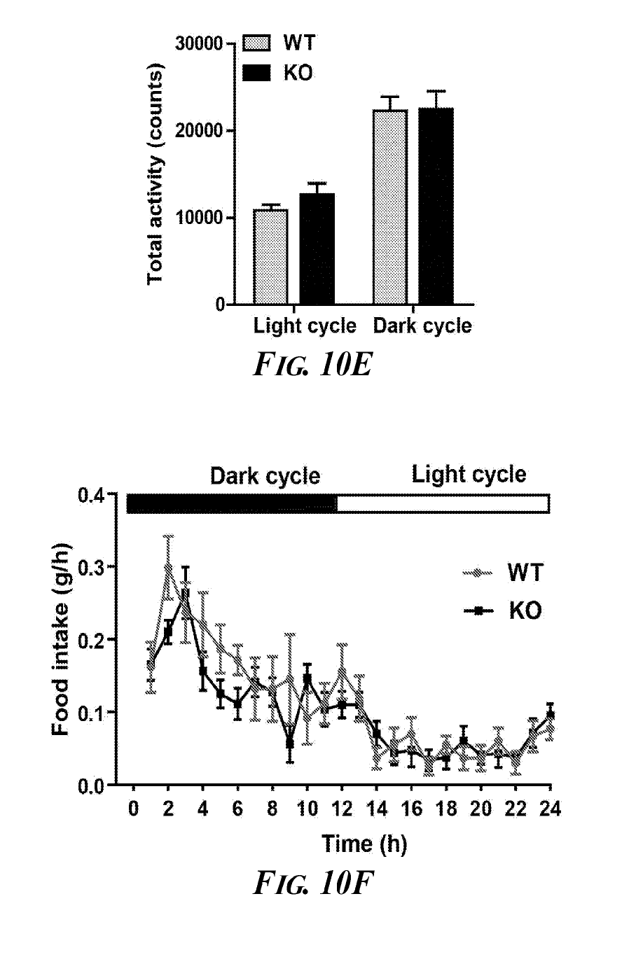

Inflammatory and fibrotic states of adipose tissue—HFD-induced obesity is known to result in adipose tissue inflammation due to macrophage infiltration, as well as tissue remodeling (i.e., fibrosis), both of which compromise adipose tissue health and function and lead to dysregulated systemic glucose and lipid metabolism (Hotamisligil, 2006; Sun, et al., 2013). We sought to determine whether there is a difference in adipose tissue inflammation and fibrosis in Ctrp5 WT and KO mice in response to HFD. Histology of visceral (epididymal) adipose tissue did not reveal any differences in the size of adipocytes or the extent of immune cell infiltration (FIG. 9A). Consistent with the histology, no significant differences were seen in the expression of macrophage marker genes (F4/80 and Cd11) in both visceral (epididymal) and subcutaneous (inguinal) white adipose tissue (FIG. 9B and FIG. 9C). We also examined the expression of fibrotic collagen genes (Col3 and Col6) and did not observe any differences between the two groups of mice (FIG. 9D and FIG. 9E). It has been shown that high-fat diet also disrupts pancreatic islet morphology and promotes kidney fibrosis (Brownlee, 2005). However, examination of tissue sections of pancreas and kidney also revealed no differences between WT and KO animals (data not shown). Since circulating adipokines produced by adipose tissue play important roles in regulating systemic insulin sensitivity, we measured serum levels of leptin, adiponectin, IL-6, and TNF-α and did not see any differences between WT and KO mice (FIG. 9F, FIG. 9G, FIG. 9H, and FIG. 9I).

Given that CTRP5 is structurally related to other CTRP family members, such as adiponectin, which is known to have important metabolic functions (Peterson, et al., 2012; Peterson, et al., 2013; Peterson, et al., Am J Physiol Regul Integr Com Physiol, 2013; Wei, et al., 2014; Wei, et al., 2012; Kadowaki, et al., 2006), we sought to determine whether deletion of the Ctrp5 gene could lead to a compensatory upregulated expression of adiponectin and/or other CTRPs. However, in both visceral (epididymal) and subcutaneous (inguinal) fat depots, the expression of Ctrp1, Ctrp3, Ctrp9, Ctrp12, and adiponectin (adipoq) was not different between WT and KO mice (FIG. 9J and FIG. 9K).

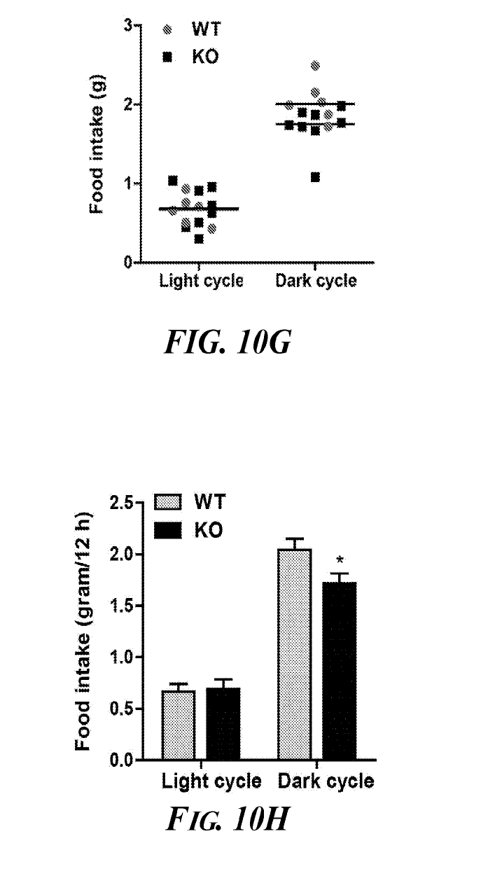

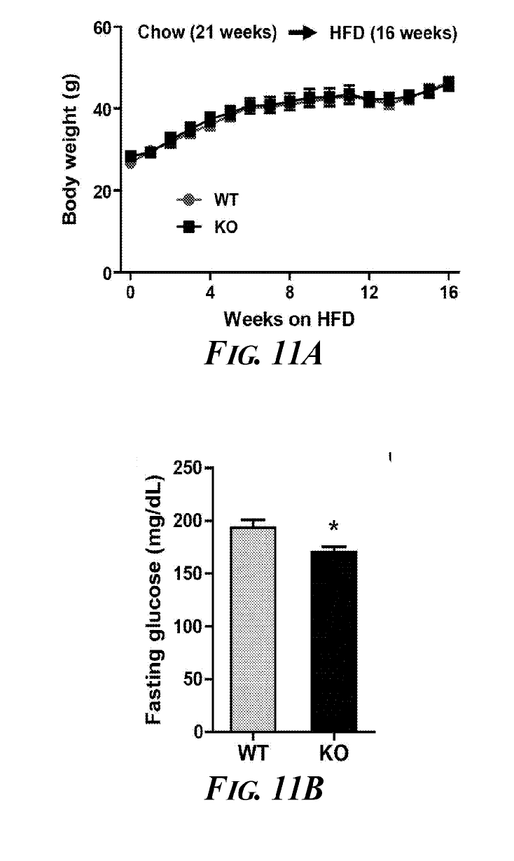

Reduced food intake in HFD-fed Ctrp5 KO mice—To determine the impact of CTRP5 deficiency on whole-body energy balance, we performed indirect calorimetry analyses on HFD-fed WT and KO mice. Both groups of mice had similar rates of oxygen consumption (VO₂), carbon dioxide production (VCO₂), respiratory exchange ratios (RER), and energy expenditure (EE) (FIG. 10A, FIG. 10B, FIG. 10C, and FIG. 10D). The physical activity levels during the light and dark phases of the light cycle were also similar between WT and KO mice (FIG. 10E). Food intake, however, was reduced in Ctrp5 KO mice relative to WT controls during the dark cycle, when mice are most active (FIG. 10F and FIG. 10G). Reduced food intake did not result in a significant change in average body weight between the two groups of mice over time (FIG. 6A).

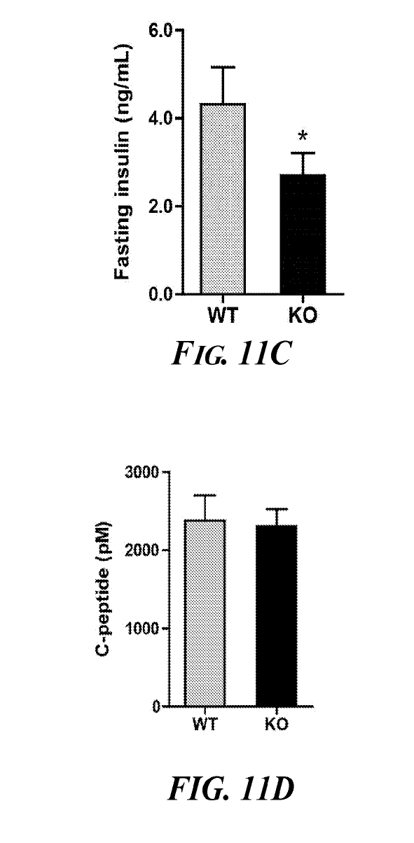

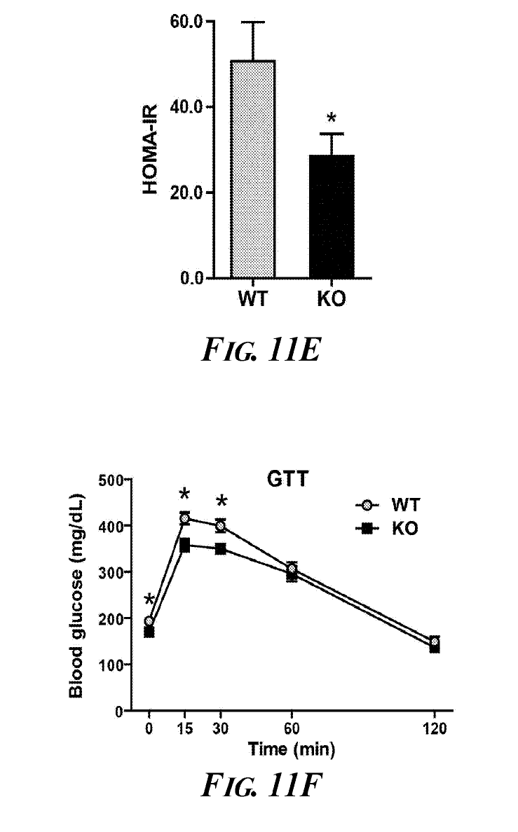

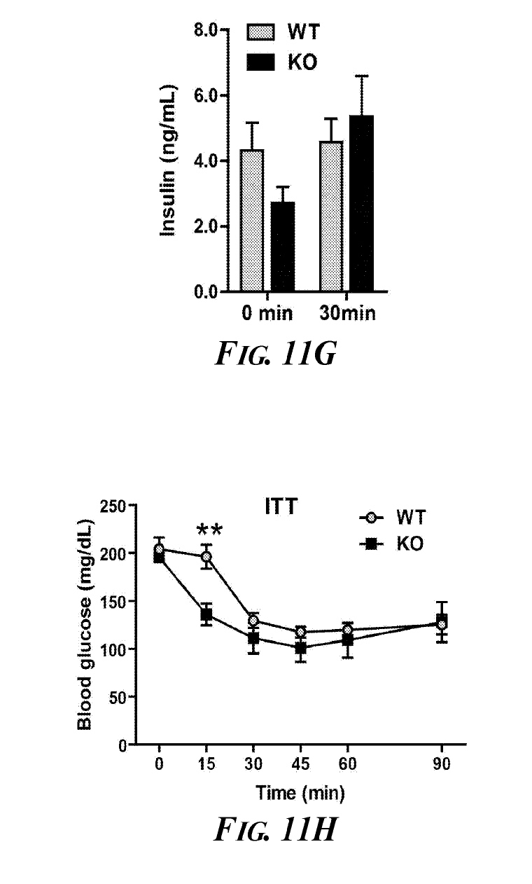

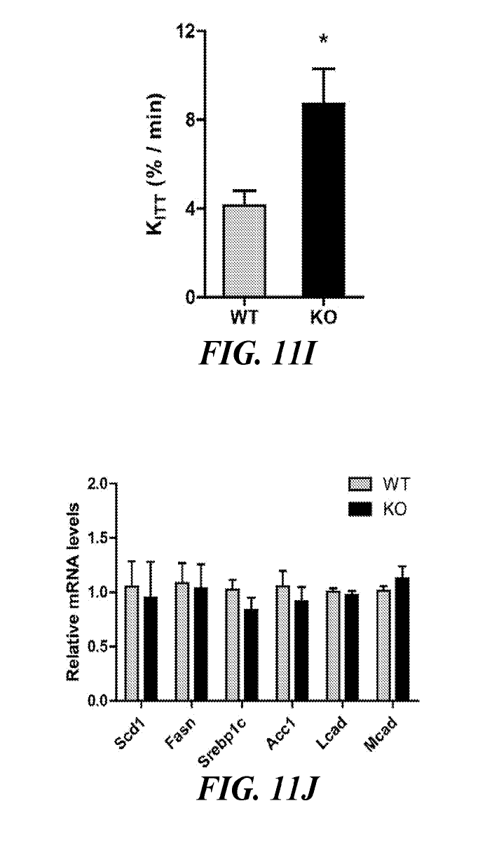

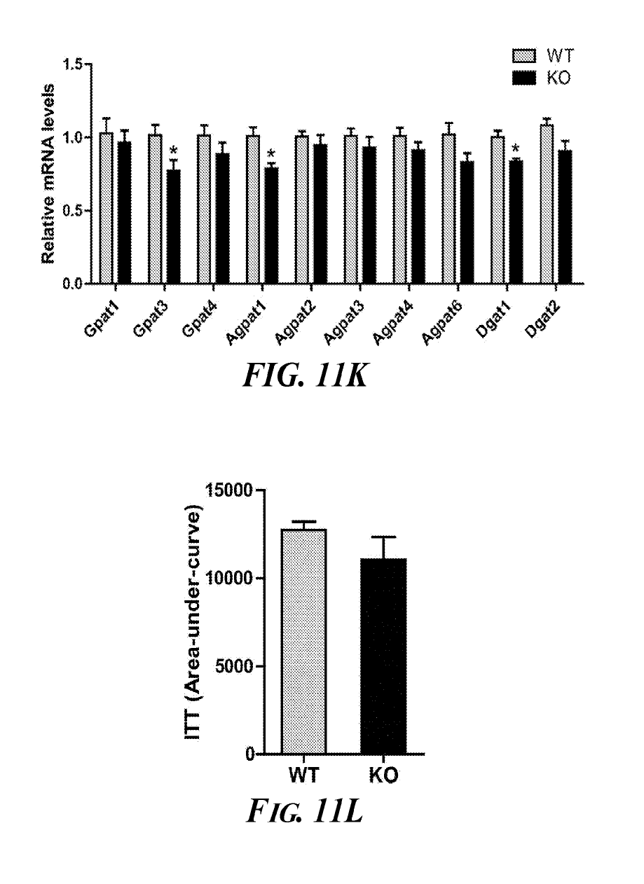

Improved metabolic phenotype in aged Ctrp5 KO mice fed a HFD—Metabolic derangement is exacerbated by age (Hildrum, et al., 2007). Therefore, we sought to address how well the aged Ctrp5 KO mice responded to metabolic stress induced by high-fat feeding. To do so, a separate cohort of WT and KO mice were initially fed a standard laboratory chow for the first 21 weeks after weaning. Thereafter, mice were switched over to a HFD for another 16 weeks. This enabled us to determine how well the aged mice (41-week-old) cope with the metabolic impacts of switching to a high-fat diet as an adult. After just one week of high-fat feeding, the WT mice gained proportionally more weight (% BW gain) than the KO animals, and the differences in percent body weight gain became more significant from weeks 6 to 12 (data not shown). However, the average body weight (in grams) was not different between the two groups of mice (FIG. 11A). In aged mice (>10 months of age) fed a HFD later in life, Ctrp5 deficiency lowered fasting blood glucose and insulin (FIG. 11B and FIG. 11C), reduced insulin resistance (FIG. 11E), and improved glucose and insulin tolerance (FIG. 11F, FIG. 11G, FIG. 11H, and FIG. 11L).

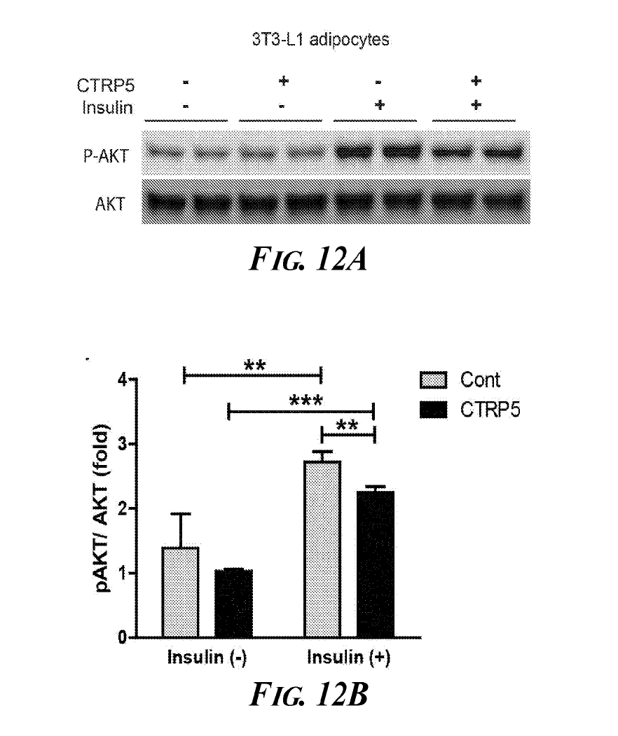

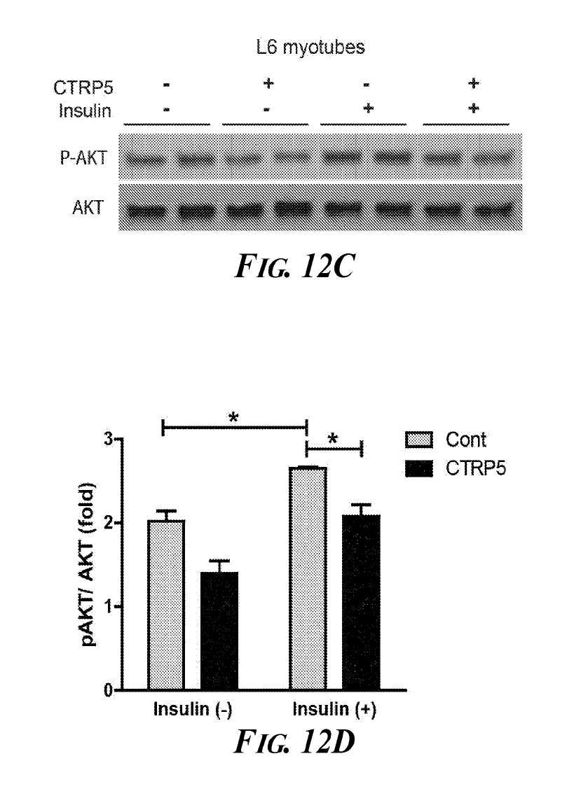

CTRP5 impairs insulin signaling in cultured adipocytes and myotubes—Since mice lacking CTRP5 had improved insulin action and reduced insulin resistance, we next addressed whether the observed in vivo metabolic phenotypes were due to the direct action of CTRP5 on cells or via an indirect mechanism. To test this, we used established cell culture models of mouse adipocytes (3T3-L1) and rat myotubes (L6). As expected, insulin robustly stimulated the phosphorylation of protein kinase B/Akt in adipocytes and myotubes (FIG. 12). However, when cells were pre-treated with conditioned media containing recombinant CTRP5, insulin-stimulated Akt phosphorylation was attenuated compared to cells treated with control conditioned media (FIG. 12), suggesting that recombinant CTRP5 can act on cells to negatively modulate insulin signaling.

### Discussion

In the initial description of mouse CTRP5, we showed that the transcript is widely expressed by a variety of tissues with the highest levels in the eye (Wong, et al., 2004; Wong, et al., 2008). Expression of human CTRP5, however, has only been examined in ocular tissue in the context of disease-causing mutations that result in L-ORD (Hayward, et al., 2003; Ayyagari, et al., 2005). While the functional capabilities of CTRP5 remain largely unclear, we report here several lines of in vivo evidence to establish, for the first time, the metabolic function of CTRP5 in peripheral tissues. Consistent with a metabolic role for CTRP5, CTRP5 expression is highly responsive to acute and chronic alterations in metabolic state. Whereas refeeding following a fast reduced the expression of Ctrp5 in adipose, skeletal muscle, and liver relative to the fasted state, its expression was unchanged in the refed state in hypothalamus. As a negative regulator of insulin action, reduced Ctrp5 expression in these tissues may enhance insulin sensitivity in the refed state. We show that different diets also modulate the expression of Ctrp5 in peripheral tissues. While HFD significantly upregulates the expression of Ctrp5 in the visceral (epididymal) white adipose tissue, a ketogenic diet not only upregulated Ctrp5 expression in eWAT but also downregulated the expression of Ctrp5 in the heart. In rodents, a ketogenic diet has been shown to promote hepatic insulin resistance despite reduced weight gain (Jornayvaz, et al., 2010); thus, the upregulated expression of Ctrp5 by a ketogenic diet may contribute to impaired hepatic insulin action seen in the previous study. The significance of reduced Ctrp5 expression in the heart in response to a ketogenic diet is presently unclear.

In human and rodent models of obesity, CTRP5 mRNA expression in the adipose tissue was significantly upregulated, consistent with previous studies showing increased human CTRP5 expression in obese Pima Indians (Lee, et al., 2005), as well as increased serum CTRP5 levels in genetic models (ob/ob and db/db) of obesity in rodents (Park, et al., 2009). In humans, the expression of CTRP5 in subcutaneous, but not visceral (omental), white adipose tissue is also positively correlated with BMI and is upregulated in obese individuals with or without type 2 diabetes. Although our sample size was small, an increase in CTRP5 expression in subcutaneous adipose tissue has also been reported for obese Pima Indians (Lee, et al., 2005). These observations underscore the relevance of CTRP5 to human metabolic disorders.

Previous in vitro studies in mouse C2C12 myocytes and rat L6 myotubes using either bacterially-produced recombinant rat CTRP5 (fused to a GST tag) or the truncated globular domain of human CTRP5 indicated a role for CTRP5 in ameliorating lipid-induced insulin resistance and enhancing fatty acid oxidation by activating the conserved energy-sensing AMPK signaling pathway (Park, et al., 2009; Yang, et al., 2014). In the absence of in vivo data, the physiological relevance of these in vitro findings remains uncertain. To help resolve this, we used a genetic loss-of-function approach in the present study to interrogate the metabolic function of endogenous CTRP5 in a physiological context. We show that mice lacking CTRP5, fed either control chow, HFD at weaning, or HFD later in life (beginning at 4 months), have improved insulin sensitivity. A reduction in food intake was also observed in Ctrp5 KO mice fed a HFD, but this was not sufficient to affect the average body weight of KO mice compared to WT controls. Deleting the Ctrp5 gene also did not alter whole-body metabolic rate (VO₂), physical activity, energy expenditure, or adipose tissue inflammatory and fibrotic states. Respiratory exchange ratios (RER) did not reveal any differences in fat oxidation between Ctrp5 WT and KO mice, nor did we observe any differences in skeletal muscle AMPK phosphorylation (Thr-172) and activation between WT and KO animals (data not shown). Since targeted deletion of Ctrp5 gene improved insulin action, our data suggest that CTRP5 negatively regulates glucose metabolism in vivo, contrary to the previously suggested positive role of CTRP5 based on in vitro studies (Park, et al., 2009). Our results thus underscore the importance of using a genetic approach to help establish the critical metabolic function of CTRP5 in an intact organism.

Using in vitro cell culture models of adipocytes and myotubes, we show that CTRP5 can attenuate insulin signaling, suggesting that the in vivo phenotypes we observed in KO mice are likely due to the effects of CTRP5 on peripheral tissues. In our in vitro studies, full-length recombinant CTRP5 was made in mammalian HEK293 cells, thus ensuring proper posttranslational modifications of CTRP5 and the assembly of higher-order structures likely to be important for the biological function of the protein. The differences between our findings and those of Park et al. (Park, et al., 2009) may be attributable to differences between bacterially-produced GST-fusion and truncated protein versus mammalian-produced full-length CTRP5.

Metabolic regulation in vivo is a complex and robust process largely due to functional redundancy and compensation. CTRP5 belongs to the C1q family of proteins that includes adiponectin (Scherer, et al., 1995) and fourteen other related CTRP family members (Seldin, et al., 2014; Wong, et al., 2004; Seldin, et al., 2012; Wei, et al., 2012; Wei, et al., 2011; Wei, et al., 2013; Wong, et al., 2009; Wong, et al., 2008; Byerly, et al., 2014), and these secreted proteins share common structural features including a signature C-terminal globular domain homologous to the immune complement C1q (Seldin, et al., 2014; Wong, et al., 2004). Several of the CTRP family members have been shown to play important roles in regulating glucose and/or lipid metabolism in peripheral tissues (Peterson, et al., 2012; Peterson, et al., 2013; Peterson, et al., Am J Physiol Regul Integr Comp Physiol, 2013; Peterson, et al., 2010; Wei, et al., 2012), as well as having a central role in modulating food intake (Byerly, et al., 2014; Byerly, et al., 2013; Wei, et al., Am J. Physiol Endocrinol Metab, 2014). Recent in vitro studies in 3T3-L1 adipocytes using bacterially produced recombinant protein suggest that CTRP5 can inhibit the secretion of adiponectin, an insulin-sensitizing adipokine (Schmid, et al., 2013). In Ctrp5 KO mice, circulating levels of adiponectin were not different compared to WT controls, suggesting that the improved insulin action seen in the Ctrp5 KO mice is not due to a compensatory upregulated expression of adiponectin or related CTRP family members with known metabolic functions in vivo. Rather, our data suggest a distinct metabolic role for CTRP5. While other CTRPs have been shown to play positive and beneficial roles in modulating glucose and fatty acid metabolism (Peterson, et al., 2012; Peterson, et al., 2013; Peterson, et al., Am J Physiol Regul Integr Comp Physiol, 2013; Peterson, et al., 2010; Wei, et al., 2012; Enomoto, et al., 2011), CTRP5 appears to serve as a negative regulator of insulin sensitivity and glucose metabolism. The distinct functions of different CTRP family members is consistent with their remarkable and high degree of conservation throughout vertebrate evolution (Seldin, et al., 2014).

In the Ctrp5 KO mice fed a HFD, hepatic TG levels were reduced and, accordingly, liver histology also indicated reduced liver steatosis when compared to WT littermate controls. However, the expression of genes involved in de novo lipogenesis, fat oxidation, and triglyceride synthesis was not different between WT and Ctrp5 KO animals. Notably, however, we examined only the mRNA expression of the enzymes and not their protein levels or enzymatic activity. Since the HFD-fed Ctrp5 KO mice had reduced fasting insulin and enhanced insulin sensitivity, the decrease in hepatic TG levels seen in the CTRP5 deficient mice may be a consequence of improvements in systemic insulin sensitivity (Brown, et al., 2008). Lipid accumulation in hepatocytes has been associated with hepatic insulin resistance (Samuel, et al., 2010; Kotronen, et al., 2008; Kotronen, et al., 2007; Sunny, et al., 2011) but the causal relationship between these two processes remains unclear (Samuel, et al., 2010; Cohen, et al., 2011; Farese, et al., 2012; Nagle, et al., 2009). The mechanistic link between hepatic steatosis and insulin resistance remains to be fully established.

A dominant missense mutation (S163R) in the globular C1q domain of CTRP5 causes L-ORD in humans (Hayward, et al., 2003; Ayyagari, et al., 2005; Subrayan, et al., 2005). L-ORD appears to be rare and affects individuals in the fifth and sixth decades of life (Kuntz, et al., 1996; Milam, et al., 2000). A total of close to 50 individuals with L-ORD carrying the S163R mutation in the CTRP5 gene have thus far been identified; no metabolic parameters (e.g., BMI and fasting blood glucose) for these individuals have been reported (Hayward, et al., 2003; Ayyagari, et al., 2005; Subrayan, et al., 2005). Interestingly, two of the Ctrp5 S163R knock-in mouse models have contrasting phenotypes; one largely phenocopies the human retinal defects (Chavali, et al., 2011), while the other has no retinal abnormalities up to two years of age (Shu, et al., 2011). The differences between the two groups were attributed to dissimilar genetic backgrounds of the animals. Since metabolic parameters were not included (Chavali, et al., 2011; Shu, et al., 2011), we do not know if these single point mutation knock-in mice (either heterozygous or homozygous for the S163R allele) have improved insulin sensitivity comparable to the homozygous KO mice reported in the present study, in which the entire Ctrp5 gene was removed. In the case where the S163R knock-in mice developed L-ORD, overt retinal defects appeared between 12-21 months of age (Chavali, et al., 2011). Since all of our studies were conducted using Ctrp5 KO mice that are younger (between 4-10 months of age), and the retinal histology of 10-month old WT and KO mice revealed no apparent differences (data not shown), we assumed that the Ctrp5-null animals had normal retinal function within the study period.

Indeed, in Mexican cavefish (Astyanax mexicanus) that live in permanent darkness, the eye degenerates and the cavefish are blind (Jeffrey, 2001). However, its genome retains the CTRP5 gene (McGaugh, et al., 2014) (GenBank accession number: XM_007258879). The predicted A. mexicanus CTRP5 transcript (XP_007258941) is 61% identical to human CTRP5, comparable to the 65% identity between zebrafish and human CTRP5. The retention of CTRP5 gene in blind Mexican cavefish further suggests that the encoded secreted protein has a hormonal role in peripheral tissue in addition to its role in the retina.

Our results support a metabolic function for Ctrp5. The genetic loss-of-function studies described herein establish CTRP5 as a negative regulator of glucose metabolism and insulin sensitivity, and our results provide a mechanistic link between increased adipose expression of CTRP5 and impaired glucose homeostasis in obesity. Inhibiting CTRP5 action may prove valuable in improving insulin resistance associated with obesity and future studies using KO mice may uncover additional physiological roles for this secreted protein in both normal and disease states.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Albertie, J. G., and Zimmet, P. Z. (1998) Definition, diagnosis and classification of diabetes mellitus and its complications. Part 1: diagnosis and classification of diabetes mellitus provisional report of a WHO consultation. Diabet Med 15, 539-553
Ayyagari, R., Mandal, M. N., Karoukis, A. J., Chen, L., McLaren, N. C., Lichter, M., Wong, D. T. Hitchcock, P. F., Caruso, R. C., Moroi, S. E., Maumenee, I. H., and Sieving, P. A. (2005) Late-onset macular degeneration and long anterior lens zonules result from a CTRP5 gene mutation. Invest Ophthalmol Vis Sci 46, 3363-3371
Bonora, E., Manicardi, V., Zavaroni, I., Coscelli, C., Butturini, U. (1987) Relationships between insulin secretion, insulin metabolism and insulin resistance in mild glucose intolerance. Diabete Metab 13, 116-121
Brown, M. S., and Goldstein, J. L. (2008) Selective versus total insulin resistance: a pathogenic paradox. Cell Metab 7, 95-96
Bligh, E. G., Dyer, W. J. (1959) A rapid method of total lipid extraction and purification. Can J Biochem Physiol 37, 911-917
Brownlee, M. (2005) The pathobiology of diabetic complications: a unifying mechanism. Diabetes 54, 1615-1625
Byerly, M. S., Petersen, P. S., Ramamurthy, S., Seldin, M. M., Lei, X., Provost, E., Wei, Z., Ronnett, G. V., and Wong, G. W. (2014) C1q/TNF-related Protein 4 (CTRP4) Is a Unique Secreted Protein with Two Tandem C1q Domains That Functions in the Hypothalamus to Modulate Food Intake and Body Weight. J Biol Chem 289, 4055-4069
Byerly, M. S., Swanson, R., Wei, Z., Seldin, M. M., McCulloh, P. S., and Wong, G. W. (2013) A Central Role for C1q/TNF-Related Protein 13 (CTRP13) in Modulating Food Intake and Body Weight PLoS One 8, e62862
Cacho, J., Sevillano, J., de Castro, J, Herrera, E., Ramos, M. P. (2008) Validation of simple indexes to assess insulin sensitivity during pregnancy in Wistar and Sprague-Dawley rats. Am J Physiol Endocrinol Metab 295, E1269-E1276
Chavali, V. R., Khan, N. W., Cukras, C. A., Bartsch, D. U., Jablonski, M. M., and Ayyagari, R. (2011) A CTRP5 gene S163R mutation knock-in mouse model for late-onset retinal degeneration. Hum Mol Genet 20, 2000-2014
Choi, H. Y., Park, J. W., Lee, N., Hwang, S. Y., Cho, G. J., Hong, H. C., Yoo, H. J., Hwang, T. G., Kim, S. M., Baik, S. H., Park, K. S., Youn, B. S., and Choi, K. M. (2013) Effects of a combine aerobic and resistance exercise program on C1q/TNF-related protein-3 (CTRP-3) and CTRP-5 levels. Diabetes Care 23, 3321-3327
Cohen, J. C., Horton, J. D., and Hobbs, H. H. (2011) Human fatty liver disease: old questions and new insights. Science 332, 1519-1523
Dinculescu, A., Min, S. H., Dyka, F. M., Deng, W. T., Stupay, R. M., Chido, V., Smith, W. C., Hauswirth, W. W. (2015) Pathological Effects of Mutant C1QTNF5 (S163R) Expression in Murine Retinal Pigment Epithelium. Invest Ophthalmol Vis Sci 56, 6971-6980
Ellis, J. M., Bowman, C. E., and Wolfgang, M. J. (2015) Metabolic and Tissue-Specific Regulation of Acyl-CoA Metabolism. PLoS One 10, e0116587
Enomoto, T., Ohashi, K., Shibata, R., Higuchi, A., Maruyama, S., Izumiya, Y., Walsh, K., Murohara, T., and Ouchi, N. (2011) Adipolin/C1qdc2/CTRP12 functions as an adipokine that improves glucose metabolism. J Biol Chem 286, 34552-34558
Farese, R. V., Jr., Zechner, R., Newgard, C. B., and Walther, T. C. (2012) The problem of establishing relationships between hepatic steatosis and hepatic insulin resistance. Cell Metab 15, 570-573
Hayward, C., Shu, X., Cideciyan, A. V., Lennon, A., Barran, P., Zareparsi, S., Sawyer, L., Hendry, G., Dhillon, B., Milam, A. H., Luthert, P. J., Swaroop, A., Hastie, N. D., Jacobson, S. G., and Wright, A. F. (2003) Mutation in a short-chain collagen gene, CTRP5, results in extracellular deposit formation in late-onset retinal degeneration: a genetic model for age-related macular degeneration. Hum Mol Genet 12, 2657-2667
Hildrum, B. Mykletun, A., Hole, T., Midthjell, K., and Dahl, A. A. (2007) Age-specific prevalence of the metabolic syndrome defined by the International Diabetes Federation and the National Cholesterol Education Program: the Norwegian HUNT 2 study. BMC public health 7, 220
Hotamisligil, G. S. (2006) Inflammation and metabolic disorders. Nature 444, 860-867
Jeffrey W. R. (2001) Cavefish as a model system in evolutionary developemental biology. Dev Biol 231, 1-12
Jornayvaz, F. R., Jurczak, M. J., Lee, H. Y., Birkenfeld, A. L., Frederick, D. W., Zhang, D., Zhang, X. M., Samuel, V. T., and Shulam, G. I. (2010) A high-fat, ketogenic diet causes hepatic insulin resistance in mice, despite increasing energy expenditure and preventing weight gain. Am J Physiol Endocrinol Metab 299, E808-815
Kadowaki, T., Yamauchi, T., Kubota, N., Hara, K., Ueki, K., and Tobe, K. (2006) Adiponectin and adiponectin receptors in insulin resistance, diabetes, and the metabolic syndrome. J Clin Invest 116, 1784-1792
Kennedy, A. R., Pissios, P., Otu, H., Roberson, R., Xue, B., Asakura, K., Furukawa, N., Marino, F. E., Liu, F. F., Kahn, B. B., Libermann, T. A., and Maratos-Flier, E. (2007) A high-fat, ketogenic diet induces a unique metabolic state in mice. Am J Physiol Endocrinol Metab 292, E1724-1739
Kotronen, A., Seppala-Lindroos, A., Bergholm, R., and Yki-Jarvinen, H. (2008) Tissue specificity of insulin resistance in humans: fat in the liver rather than muscle is associated with features of the metabolic syndrome. Diabetologia 51, 130-138
Kotronen, A., Vehkavaara, S., Seppala-Lindroos, A., Bergholm, R., and Yki-Jarvinen, H. (2007) Effect of liver fat on insulin clearance. Am J Physiol Endocrinol Metab 293, E1709-1715
Kuntz, C. A., Jacobson, S. G., Cideciyan, A. V., Li, Z. Y., Stone, E. M., Possin, D., and Milam, A. H. (1996) Sub-retinal pigment epithelial deposits in a dominant late-onset retinal degeneration. Invest Ophthalmol Vis Sci 37, 1772-1782
Larkin, M. A., Blackshields, G., Brown, N. P., Chenna, R.., McGettigan, P. A., McWilliam, H., Valentin, F., Wallace, I. M., Wilm, A., Lopez, R., Thompson, J. D., Gibson, T. J., and Higgins, D. G. (2007) Clustal W and Clustal X version 2.0 Bioinformatics 23, 2947-2948
Lee, S., Muniyappa, R., Yan, X., Chen, H., Yue, L. Q., Hong, E. G., Kim, J. K., Quon, M. J. (2008) Comparison between surrogate indexes of insulin sensitivity and resistance and hyperinsulinemic euglycemic clamp estimates in mice. Am J Physiol Endocrinol Metab 294, E261-270
Lee, Y. H., Nair, S., Rousseau, E., Allison, D. B., Page, G. P., Tataranni, P. A., Bogardus, C., and Permana, P. A. (2005) Microarray profiling of isolated abdominal subcutaneous adipocytes from obese vs non-obese Pima Indians: increased expression of inflammation-related genes. Diabetologia 45, 1776-1783
Lim, S. Choi, S. H., Koo, B. K., Kang, S. M., Yoon, J. W., Jang, H. C., Choi, S. M., Lee, M. G., Lee, W., Shin, H., Kim, Y. B., Lee, H. K., and Park, K. S. (2012) Effects of aerobic exercise training on C1q tumor necrosis factor alpha-related protein isoform 5 (myonectin): associated with insulin resistance and mitochondrial DNA density in women. J Clin Endocrinol Metab 97, E88-93
Mandal, M. N., Vasireddy, V., Reddy, G. B., Wang, X., Moroi, S. E., Pattnaik, B. R., Hughes, B. A., Heckenlively, J. R., Hitchcock, P. F., Jablonski, M. M., and Ayyagari, R. (2006) CTRP5 is a membrane-associated and secretory protein in the RPE and ciliary body and the S163R mutation of CTRP5 impairs its secretion. Invest Ophthalmol Vis Sci 47, 5505-5513
Matthews, D. R., Hosker, J. P., Rudenski, A., S., Naylor, B. A., Treacher, D. F., and Turner, R. C. (1985) Homeostasis model assesment: insulin resistance and beta-cell function from fasting plasma glucose and insulin concentrations in man. Diabetologia 28, 412-419
McGaugh, S. E., Gross, J. B., Aken, B., Blin, M., Borowsky, R., Chalopin, D., Hinaux, H., Jeffrey, W. R., Keene, A., Ma, L., Minx, P., Murphy, D., O'Quin, K. E., Retaux, S., Rohner, N., Searle, S. M., Stahl, B. A., Tabin, C., Volff, J. N., Yoshizawa, M., and Warren, W. C. (2014) The cavefish genome reveals candidate genes for eye loss. Nature communications 5, 5307
Milam, A. H., Curcio, C. A., Cideciyan, A. V., Saxena, S., John, S. K., Kruth, H. S., Malek, G., Heckenlively, J. R., Weleber, R. G., and Jacobson, S. G. (2000) Dominant late-onset retinal degeneration with regional variation of sub-retinal pigment epithelium deposits, retinal function, and photoreceptor degeneration. Ophthalmology 107; 2256-2266
Nagle, C. A., Klett, E. L., and Coleman, R. A. (2009) Hepatic triacylglycerol accumulation and insulin resistance. J Lipid Res 50 Suppl, S74-79
Park, S. Y., Choi, J. H., Ryu, H. S., Pak, Y. K., Park, K. S., Lee, H. K., and Lee, W. (2009) C1q tumor necrosis factor alpha-related protein isoform 5 is increased in mitochondrial DNA-depleted myocytes and activates AMP-activated protein kinase. J Biol Chem 284, 27780-27789
Peterson, J. M., Aja, S., Wei, Z., and Wong, G. W. (2012) C1q/TNF-related protein-1 (CTRP1) enhances fatty acid oxidation via AMPK activation and ACC inhibition. J. Biol Chem 287, 1576-1587
Peterson, J. M., Seldin, M. M., Wei, Z., Aja, S., and Wong, G. W. (2013) CTRP3 attenuates diet-induced hepatic steatosis by regulating triglyceride metabolism. Am J Physiol Gastrointest Liver Physiol 305, G214-224
Peterson, J. M., Wei, Z., Seldin, M. M., Byerly, M. S., Aja, S., and Wong, G. W. (2013) CTRP9 transgenic mice are protected from diet-induced obesity and metabolic dysfunction. Am J Physiol Regul Integr Comp Physiol 305, R522-533
Peterson, J. M., Wei, Z., and Wong, G. W. (2010) C1q/TNF-related Protein-3 (CTRP3), a Novel Adipokine That Regulates Hepatic Glucose Output. J. Biol Chem 285, 39691-39701
Samuel, V. T., Petersen, K. F., and Shulman, G. I. (2010) Lipid-induced insulin resistance: unravelling the mechanism. Lancet 375, 2267-2277
Scherer, P. E., Williams, S., Fogliano, M., Baldini, G., and Lodish, H. F. (1995) A novel serum protein similar to C1q produced exclusively in adipocytes. J Biol Chem 270, 26746-26749
Schmid, A., Kopp, A., Aslanidis, C., Wabitsch, M., Muller, M., and Schaffler, A. (2013) Regulation and Function of C1q/TNF-related Protein-5 (CTRP-5) in the Context of Adipocyte Biology. Exp Clin Endocrinol Diabetes 121, 310-317
Schmittgen, T. D., and Livak, K. J. (2008) Analyzing real-time PCR data by the comparative C(T) method Nat Protoc 3, 1101-1108
Seldin, M. M., Lei, X., Tan, S. Y., Stanson, K. P., Wei, Z., and Wong, G. W. (2013) Skeletal muscle-derived myonectin activates the mTOR pathway to suppress autophagy in liver. J. Biol Chem 289, 36073-36082
Seldin, M. M., Peterson, J. M., Byerly, M. S., Wei, Z., and Wong, G. W. (2012) Myonectin (CTRP15), a novel myokine that links skeletal muscle to systemic lipid homeostasis. J. Biol Chem 287, 11968-11980
Seldin, M. M., Tan, S. Y., and Wong, G. W. (2014) Metabolic function of the CTRP family of hormones. Rev Endocr Metab Disord 15, 111-123
Shu, X., Luhmann, U. F., Aleman, T. S., Barker, S. E., Lennon, A., Tulloch, B., Chen, M., Xu, H., Jacobson, S. G., Ali, R., and Wright, A. F. (2011) Characterisation of a C1qtnf5 Ser163Arg knock-in mouse model of late-onset retinal macular degeneration. PLoS One 6, e27433
Shu, X., Tulloch, B., Lennon, A., Hayward, C., O′Connell, M., Cideciyan, A. V., Jacobson, S. G., and Wright, A. F. (2006) Biochemical characterisation of the C1QTNF5 gene associated with late-onset retinal degeneration. A genetic model of age-related macular degeneration. Adv Exp Med Biol 572, 41-48
Shu, X., Tulloch, B., Lennon, A., Vlachantoni, D., Zhou, X., Hayward, C., and Wright, A. F. (2006) Disease mechanisms in late-onset retinal macular degeneration associated with mutation in C1QTNF5. Hum Mol Genet 15, 1680-1689
Soumplis, V., Sergouniotis, P. I., Robson, A. G., Michaelides, M., Moore, A. T., Holder, G. E., and Webster, A. R. (2013) Phenotypic findings in C1QTNF5 retinopathy (late-onset retinal degeneration). Acta Ophthalmol 91, e191-195
Subrayan, V., Morris, B., Armbrecht, A. M., Wright, A. F., and Dhillon, B. (2005) Long anterior lens zonules in late-onset retinal degeneration (L-ORD). Am J Ophthamol 140, 1127-1129
Sun, K., Tordjman, J., Clement, K., and Scherer, P. E. (2013) Fibrosis and adipose tissue dysfunction. Cell Metab 18, 470-477
Sunny, N. E., Parks, E. J., Browning, J. D., and Burgess, S. C. (2011) Excessive hepatic mitochondrial TCA cycle and gluconeogenesis in humans with nonalcoholic fatty liver disease. Cell Metab 14, 804-810
Sun, K., Tordjman, J., Clement, K., and Scherer, P. E. (2013) Fibrosis and adipose tissue dysfunction. Cell Metab 18, 470-477
Sunny, N. E., Parks, E. J., Browning, J. D., and Burgess, S. C. (2011) Excessive hepatic mitochondrial TCA cycle and gluconeogenesis in humans with nonalcoholic fatty liver disease. Cell Metab 14, 804-810
Tu, X., and Palczewski, K. (2014) The macular degeneration-linked C1QTNF5 (S163) mutation causes higher-order structural rearrangements. J Struct Biol
Vincent, A., Munier, F. L., Vandenhoven, C. C., Wright, T., Westall, C. A., and Heon, E. (2012) The characterization of retinal phenotype in a family with C1QTNF5-related late-onset retinal degeneration. Retina 32, 1643-1651
Wei, Z., Lei, X., Petersen, P. S., Aja, S., and Wong, G. W. (2014) Targeted deletion of C1q/TNF-related protein 9 (CTRP9) increases food intake, decreases insulin sensitivity, and promotes hepatic steatosis in mice. Am J Physiol Endocrinol Metab 306, E779-E790
Wei, Z., Peterson, J. M., Lei, X., Cebotaru, L., Wolfgang, M. J., Baldeviano, G. C., and Wong, G. W. (2012) C1q/TNF-related protein-12 (CTRP12), a novel adipokine that improves insulin sensitivity and glycemic control in mouse models of obesity and diabetes. J Biol Chem 287, 10301-10315
Wei, Z., Peterson, J. M., and Wong, G. W. (2011) Metabolic regulation by C1q/TNF-related protein-13 (CTRP13): activation OF AMP-activated protein kinase and suppression of fatty acid-induced JNK signaling. J Biol Chem 286, 15652-15665
Wei, Z., Seldin, M. M., Natarajan, N., Djemal, D. C., Peterson, J. M., and Wong, G. W. (2013) C1q/Tumor Necrosis Factor-related Protein 11 (CTRP11), a Novel Adipose Stroma-derived Regulator of Adipogenesis. J Biol Chem 288, 10214-10229
Wong, G. W., Krawczyk, S. A., Kitidis-Mitrokostas, C., Ge, G., Spooner, E., Hug, C., Gimeno, R., and Lodish, H. F. (2009) Identification and characterization of CTRP9, a novel secreted glycoprotein, from adipose tissue that reduces serum glucose in mice and forms heterotrimers with adiponectin. FASEB J 23, 241-258
Wong, G. W., Krawczyk, S. A., Kitidis-Mitrokostas, C., Revett, T., Gimeno, R., and Lodish, H. F. (2008) Molecular, biochemical and functional characterizations of C1q/TNF family members: adipose-tissue-selective expression patterns, regulation by PPAR-gamma agonist, cysteine-mediated oligomerizations, combinatorial associations and metabolic functions. Biochem J416, 161-177
Wong, G. W., Wang, J., Hug, C., Tsao, T. S., and Lodish, H. F. (2004) A family of Acrp30/adiponectin structural and functional paralogs. Proc Natl Acad Sci USA 101, 10302-10307
Yang, W. M., and Lee, W. (2014) CTRP5 ameliorates palmitate-induced apoptosis and insulin resistance through activation of AMPK and fatty acid oxidation. Biochem Biophys Res Commun 452, 715-721

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method of improving insulin sensitivity in a cell, tissue, or subject, the method comprising administering to a cell, tissue, or subject an effective amount of an agent that decreases the expression level and/or activity of C1q and tumor necrosis factor related protein 5 (CTRP5), thereby improving insulin sensitivity in the cell, tissue, or subject.

2. The method of claim 1, wherein the expression level and/or activity of CTRP5 is decreased in the cell, tissue, or subject.

3. The method of claim 1, wherein the cell is selected from the group consisting of an adipocyte, a myocyte, a hepatocyte and combinations thereof.

4. The method of claim 1, wherein the tissue comprises a peripheral tissue.

5. The method of claim 1, wherein the tissue is selected from the group consisting of adipose tissue, skeletal muscle, liver, and combinations thereof.

6. The method of claim 6, wherein the adipose tissue comprises subcutaneous white adipose tissue.

7. The method of claim 1, wherein the subject:
(i) has a metabolic disorder or is at risk of developing a metabolic disorder;
(ii) is obese or at risk of becoming obese;
(iii) has hepatic steatosis; and/or
(iv) has diabetes or is at risk of developing diabetes.

8. The method of claim 1, wherein the subject is refed in a fasted state.

9. The method of claim 1, further comprising administering to the subject an effective amount of an anti-diabetic agent and/or an appetite suppressant agent.

10. A method of treating a metabolic disorder in a subject in need thereof, the method comprising administering to the subject an effective amount of an agent that decreases the expression level and/or activity of CTRP5, wherein the agent reduces insulin resistance, improves glucose homeostasis, reduces hepatic triglyceride levels, and/or reduces food intake in the subject, thereby treating a metabolic disorder in the subject.

11. The method of claim 10, wherein the expression level and/or activity of CTRP5 is decreased in a cell or tissue of the subject.

12. The method of claim 10, wherein the cell is selected from the group consisting of an adipocyte, a myocyte, a hepatocyte and combinations thereof

13. The method of claim 10, wherein the tissue comprises a peripheral tissue.

14. The method of claim 10, wherein the tissue is selected from the group consisting of adipose tissue, skeletal muscle, liver, and combinations thereof

15. The method of claim 14, wherein the adipose tissue comprises subcutaneous white adipose tissue.

16. The method of claim 10, wherein the subject:
(i) has a metabolic disorder or is at risk of developing a metabolic disorder;
(ii) is obese or at risk of becoming obese;
(iii) has hepatic steatosis; and/or
(iv) has diabetes or is at risk of developing diabetes.

17. The method of claim 10, wherein the subject is refed following a fast.

18. The method of claim 10, further comprising administering to the subject a high fat diet.

19. The method of claim 18, wherein the subject is administered a high fat diet in a fasted state.

20. The method of claim 10, further comprising administering to the subject an effective amount of an anti-diabetic agent and/or an appetite suppressant agent.


SEQUENCE LISTING (61 sequences)

SEQ ID NO: 1 - DNA, 23 bp, Artificial Sequence
  Genotyping primer
cagaaaccctgatgcctctactc

SEQ ID NO: 2 - DNA, 23 bp, Artificial Sequence
  Genotyping primer
ggagaaattaggagccgcagaag

SEQ ID NO: 3 - DNA, 22 bp, Artificial Sequence
  Genotyping primer
ggtaaactggctcggattaggg

SEQ ID NO: 4 - DNA, 22 bp, Artificial Sequence
  Genotyping primer
ttgactgtagcggctgatgttg

SEQ ID NO: 5 - DNA, 18 bp, Homo sapiens
cctcgcctttgccgatcc

SEQ ID NO: 6 - DNA, 18 bp, Homo sapiens
cgcggcgatatcatcatc

SEQ ID NO: 7 - DNA, 23 bp, Homo sapiens
cccacctgcaaagtgagctcatg

SEQ ID NO: 8 - DNA, 22 bp, Homo sapiens
ctagtcattcacaatattccag

SEQ ID NO: 9 - DNA, 20 bp, Artificial Sequence
  Forward primer sequence
gcaattattccccatgaacg

SEQ ID NO: 10 - DNA, 20 bp, Artificial Sequence
  Reverse primer sequence
ggcctcactaaaccatccaa

SEQ ID NO: 11 - DNA, 21 bp, Artificial Sequence
  Forward primer sequence
agtgtgacgttgacatccgta

SEQ ID NO: 12 - DNA, 22 bp, Artificial Sequence
  Reverse primer sequence
gccagagcagtaatctccttct

SEQ ID NO: 13 - DNA, 21 bp, Artificial Sequence
  Forward primer sequence
agcaggttttgaagttcaccc

SEQ ID NO: 14 - DNA, 21 bp, Artificial Sequence
  Reverse primer sequence
cagctttcccattcaccttga

SEQ ID NO: 15 - DNA, 18 bp, Artificial Sequence
  Forward primer sequence
tggagtctgagcctccgg

SEQ ID NO: 16 - DNA, 19 bp, Artificial Sequence
  Reverse primer sequence
agaagggcaagaagtggcc

SEQ ID NO: 17 - DNA, 21 bp, Artificial Sequence
  Forward primer sequence
cccagtcgtacacgtcatttt

SEQ ID NO: 18 - DNA, 21 bp, Artificial Sequence
  Reverse primer sequence
catcattctcatggtcctgct

SEQ ID NO: 19 - DNA, 21 bp, Artificial Sequence
  Forward primer sequence
gctgcggaaacttcagaaaat

SEQ ID NO: 20 - DNA, 23 bp, Artificial Sequence
  Reverse primer sequence
agagacgtgtcactcctggactt

SEQ ID NO: 21 - DNA, 20 bp, Artificial Sequence
  Forward primer sequence
ggagccatggattgcacatt

SEQ ID NO: 22 - DNA, 18 bp, Artificial Sequence
  Reverse primer sequence
ggcccgggaagtcactgt

SEQ ID NO: 23 - DNA, 23 bp, Artificial Sequence
  Forward primer sequence
tgacagactgatcgcagagaaag

SEQ ID NO: 24 - DNA, 18 bp, Artificial Sequence
  Reverse primer sequence
tggagagccccacacaca

SEQ ID NO: 25 - DNA, 21 bp, Artificial Sequence
  Forward primer sequence
tcttttcctcggagcatgaca

SEQ ID NO: 26 - DNA, 23 bp, Artificial Sequence
  Reverse primer sequence
gacctctctactcacttctccag

SEQ ID NO: 27 - DNA, 23 bp, Artificial Sequence
  Forward primer sequence
agggtttagttttgagttgacgg

SEQ ID NO: 28 - DNA, 21 bp, Artificial Sequence
  Reverse primer sequence
ccccgcttttgtcatattccg

SEQ ID NO: 29 - DNA, 19 bp, Artificial Sequence
  Forward primer sequence
caacaccatccccgacatc

SEQ ID NO: 30 - DNA, 22 bp, Artificial Sequence
  Reverse primer sequence
gtgaccttcgattatgcgatca

SEQ ID NO: 31 - DNA, 20 bp, Artificial Sequence
  Forward primer sequence
ggaggatgaagtgacccaga

SEQ ID NO: 32 - DNA, 20 bp, Artificial Sequence
  Reverse primer sequence
ccagtttttgaggctgctgt

SEQ ID NO: 33 - DNA, 20 bp, Artificial Sequence
  Forward primer sequence
tgtctggtttgagcgttctg

SEQ ID NO: 34 - DNA, 20 bp, Artificial Sequence
  Reverse primer sequence
ttctgggaagatgaggatgg

SEQ ID NO: 35 - DNA, 22 bp, Artificial Sequence
  Forward primer sequence
taagatggccttctacaacggc

SEQ ID NO: 36 - DNA, 23 bp, Artificial Sequence
  Reverse primer sequence
ccatacaggtatttgacgtggag

SEQ ID NO: 37 - DNA, 20 bp, Artificial Sequence
  Forward primer sequence
cagccaggttctacgccaag

SEQ ID NO: 38 - DNA, 22 bp, Artificial Sequence
  Reverse primer sequence
tgatgctcatgttatccacggt

SEQ ID NO: 39 - DNA, 21 bp, Artificial Sequence
  Forward primer sequence
ctgcttgcctacctgaagacc

SEQ ID NO: 40 - DNA, 21 bp, Artificial Sequence
  Reverse primer sequence
gatacggcggtataggtgctt

SEQ ID NO: 41 - DNA, 22 bp, Artificial Sequence
  Forward primer sequence
ccagtttctatgtcacctggtc

SEQ ID NO: 42 - DNA, 22 bp, Artificial Sequence
  Reverse primer sequence
gcagagtctggcattgatcttg

SEQ ID NO: 43 - DNA, 21 bp, Artificial Sequence
  Forward primer sequence
agcttgattgtcaacctcctg

SEQ ID NO: 44 - DNA, 19 bp, Artificial Sequence
  Reverse primer sequence
ccgttggtgtagggcttgt

SEQ ID NO: 45 - DNA, 20 bp, Artificial Sequence
  Forward primer sequence
ccctgagtatccaggcaagg

SEQ ID NO: 46 - DNA, 20 bp, Artificial Sequence
  Reverse primer sequence
aaggagtgggcctctagact

SEQ ID NO: 47 - DNA, 21 bp, Artificial Sequence
  Forward primer sequence
gcgctacttccgagactactt

SEQ ID NO: 48 - DNA, 20 bp, Artificial Sequence
  Reverse primer sequence
gggccttatgccaggaaact

SEQ ID NO: 49 - DNA, 21 bp, Artificial Sequence
  Forward primer sequence
ccccagtgtccttacagagtg

SEQ ID NO: 50 - DNA, 19 bp, Artificial Sequence
  Reverse primer sequence
gtgcccagagtggatgtct

SEQ ID NO: 51 - DNA, 22 bp, Artificial Sequence
  Forward primer sequence
ctggatagcctttcttctgctg

SEQ ID NO: 52 - DNA, 20 bp, Artificial Sequence
  Reverse primer sequence
gcacactgtgtccgaactca

SEQ ID NO: 53 - DNA, 20 bp, Artificial Sequence
  Forward primer sequence
gggtttccctggtcctaaag

SEQ ID NO: 54 - DNA, 20 bp, Artificial Sequence
  Reverse primer sequence
cctggtttcccattttctcc

SEQ ID NO: 55 - DNA, 20 bp, Artificial Sequence
  Forward primer sequence
gatgagggtgaagtgggaga

SEQ ID NO: 56 - DNA, 20 bp, Artificial Sequence
  Reverse primer sequence
cagcacgaagaggatgtcaa

SEQ ID NO: 57 - protein, 243 aa, Homo sapiens
MRPLLVLLLLGLAAGSPPLDDNKIPSLCPGHPGLPGTPGHHGSQGLPGRDGRDGRDGAPGAPGEKGEGGRPGLPGPRGDPGPRGEAGPAGPTGPAGECSVPPRSAFSAKRSESRVPPPSDAPLPFDRVLVNEQGHYDAVTGKFTCQVPGVYYFAVHATVYRASLQFDLVKNGESIASFFQFFGGWPKPASLSGGAMVRLEPEDQVWVQVGVGDYIGIYASIKTDSTFSGFLVYSDWHSSPVFA

SEQ ID NO: 58 - protein, 243 aa, Mus musculus
MRPLLALLLLGLVSGSPPLDDNKIPSLCPGQPGLPGTPGHHGSQGLPGRDGRDGRDGAPGAPGEKGEGGRPGLPGPRGEPGPRGEAGPMGAIGPAGECSVPPRSAFSAKRSESRVPPPADTPLPFDRVLLNEQGHYDPTTGKFTCQVPGVYYFAVHATVYRASLQFDLVKNGQSIASFFQYFGGWPKPASLSGGAMVRLEPEDQVWVQVGVGDYIGIYASIKTDSTFSGFLVYSDWHSSPVFA

SEQ ID NO: 59 - protein, 223 aa, Gallus gallus
DNKIPRQCSGQPGIPGTPGLHGGQGLPGRDGRDGRDGAMGMPGEKGEMGPPGAPGPRGETGSPGVDGVHGEKGAQGECAVAPRSAFSAKRSESRSPPLADQPIRFDVVLVNEQGHYDPTTGKFTCEVPGLYYFAVHATVYRASLQFDIMKNGHSIASFFQYYGNWPKPTSLSGGALVRLEPEDEVWVQVGVGDYIGFYASVKTDSTFTGFLVYSYWHNSAVFA

SEQ ID NO: 60 - protein, 227 aa, Gallus gallus
DNKIASSCCGMPGIPGIPGTHGNAGLPGRDGRDGKDGSPGTSGPKGDSGEPGIPGARGFPGMAGPPGVQGDKGVQGECAVPPRSAFSAKLSESRTSPIPGQPVPFGKVLINEQGHYNPETGRFNCVVPGLYYFSIHGTVFRGSLHLQLMKNGHSQASFFQLGDAKKPVGLCGGAALRLDQGDEVWVQLGDYPGLYASEGTDSVFTGFLIYSDWEPHPVFNPLANTDS

SEQ ID NO: 61 - protein, 224 aa, Danio rerio
DNKIPSLCTGSPGIPGSPGLHGSPGQPGRDGRDGRDAQPGEKGERGESGELGQPGVRGLTGDRGDPGEKGERGAPGECAVAPKSAFSAKLSEARTTPLTVGDSVRFDKVILNEQGDYNPETGRFTCRVPGVYYFAVHATVYRSSLQFDLMKNGHTVASYFQIFGNWSKPASLSGGTLVHLIPGDQVWVQMALGEYIGFYSSSKTDSTFTGFLVYSDWKNSAVFA